(19) United States
(12) Patent Application Publication
(10) Pub. No.: US 2003/0162734 A1
(43) Pub. Date: Aug. 28, 2003

(54) Modulation of DENN-MADD expression and interactions for treating neurological disorders

(76) Inventors: Carol A. Miller, San Marino, CA (US); Keith Del Villar, Los Angeles, CA (US)

Correspondence Address: BINGHAM MCCUTCHEN LLP, 18th Floor, Three Embarcadero Center, San Francisco, CA, 94111, US

(21) Appl. No.: 10/187,264

(22) Filed: Jun. 28, 2002

Related U.S. Application Data
(60) Provisional application No. 60/301,608, filed on Jun. 28, 2001.

(51) Int. Cl. A61K 48/00; A61K 31/4439; A61K 31/407

(52) U.S. Cl. 514/44; 514/341; 514/410

(57) ABSTRACT

The invention describes methods for treating neurodegenerative diseases by modulating the expression of DENN in neuronal cells. It has been observed that neurodegenerative disease states are characterized by abnormal expression of DENN. The overexpression of DENN induces cell death in neuronal cells. However, reduced expression of DENN also characterizes neural tissue affected by neurodegenerative disease. Also disclosed are methods for treating neurodegenerative diseases by inhibiting the interaction of DENN-MADD (Differentially Expressed in Normal versus Neoplastic/MAPK Activating Death Domain containing)protein, also referred to herein as DENN, with c-Jun N-terminal kinases (JNKs). The invention further describes methods for treating neurodegenerative diseases by inhibiting the interaction of DENN-MADD with the p55 tumor necrosis factor receptor I (TNFRI).

### RELATED APPLICATION DATA

This application claims priority to provisional application serial No. 60/301,608 filed Jun. 28, 2002.

### GOVERNMENT SUPPORT

The U.S. Government has certain rights in this invention pursuant to National Institutes of Health grant number 5T32-NS07149 and NIMH grant number 5R37MH39145.

### FIELD OF THE INVENTION

The present invention relates to methods for treating neurodegenerative diseases based on a newly discovered apoptotic pathway.

### BACKGROUND

A large segment of the human population is afflicted by neurodegenerative diseases, which include, inter alia, Parkinson's disease, Alzheimer's disease, Huntington's disease and amyotrophic lateral sclerosis (ALS or Lou Gehrig's disease). hi general, these neurodegenerative disorders are progressive (i.e., their symptoms are not apparent until months or more commonly years after the disease has begun), and caused by an initial reduction of neuronal function, followed by a complete loss of function upon neuronal death. In addition, these neurodegenerative disorders are characterized by the presence of protein aggregates that are believed to hamper cellular functions (e.g., neurotransmission), and may ultimately result in cell death (Sasaki et al., Am. J. Pathol., 153:1149-1155 [1998]).

Alzheimer's disease is the most common neurodegenerative disorder. Recent experimental evidence suggests that neuronal death in Alzheimer's may occur through apoptosis (Smale et al., Exp. Neurol. 133(2):225-230 [1995]; and Kim et al., Science 277:373-376 [1997]). In the postmortem brains of Alzheimer's patients, expression of the apoptosis-related transcriptional factor c-jun was colocalized within the cells that also contained DNA strand breaks characteristic of apoptosis (Anderson et al., J. Neurosci., 16:1710-1719 [1996]).

Huntington's disease is an autosomal dominant progressive neurodegenerative disorder resulting from a CAG/polyglutamine repeat expansion in the gene encoding this disease, ultimately resulting in the death of striatal neurons. The polyglutamine expansion results in the formation of insoluble, high molecular weight protein aggregates similar to those seen in Alzheimer's disease (Scherzinger et al., Cell 90:549-558 [1997]). Postmortem examination of the brains of patients suffering from Huntington's disease revealed that CAG repeat length positively correlates with the degree of DNA fragmentation within the afflicted striatum (Butterworth et al., Neurosci., 87:49-53 [1998]), indicating that neuronal degeneration observed in Huntington's disease may also occur through an apoptotic process.

Amyotrophic lateral sclerosis (ALS) is caused by a progressive degeneration of spinal cord motor neurons and results in complete paralysis, respiratory depression and death. Aggregates of ubiquitinated proteins have been observed in ALS (Kato et al., Histol. Histopathol., 14:973-989 [1999]). Recent experiments suggest that death of motor neurons in ALS may have an apoptotic component (Pasinelli et al., Proc. Natl. Acad. Sci. USA 95:15763-15768 [1998]; and Martin, J. Neuropathol. Exp. Neurol., 58:459-471 [1999]).

Parkinson's disease is the second most common neurodegenerative disorder, affecting nearly 1 million people in North America. The disease is characterized by symptoms such as muscle rigidity, tremor and bradykinesia. Parkinson's disease is associated with the progressive loss of dopamine neurons in the ventral mesencephalon of the substantia nigra (Shoulson, Science 282: 1072-1074 [1998]), which innervates the major motor-control center of the forebrain, the striatum. Although a gradual decline in the number of neurons and dopamine content of the basal ganglia is normally associated with increasing age, progressive dopamine loss is pronounced in people suffering from Parkinson's disease, resulting in the appearance of symptoms when about 70-80% of striatal dopamine and 50% of nigral dopamine neurons are lost (Dunnett and Bjorklund, supra). This loss of dopamine-producing neurons resulting in a dopamine deficiency is believed to be responsible for the motor symptoms of Parkinson's disease.

Although the cause of dopaminergic cell death remains unknown, it is believed that dopaminergic cell death is affected by a combination of necrotic and apoptotic cell death. Mechanisms and signals responsible for the progressive degeneration of nigral dopamine neurons in Parkinson's disease have been proposed (Olanow et al., Ann. Neurol., 44:S1-S196 [1998]), and include oxidative stress (from the generation of reactive oxygen species), mitochondrial dysfunction, excitotoxicity, calcium imbalance, inflammatory changes and apoptosis as contributory and interdependent factors in Parkinson's disease neuronal cell death.

In light of the selective death of dopamine producing neurons, administration of L-dihydroxyphenylalanine (L-DOPA) remains the most widely used treatment of Parkinson's disease. However, the administration of therapeutically effective doses of L-DOPA is accompanied by disabling side effects. Furthermore, in some cases, treatment with L-DOPA requires the coadministration of a peripheral DOPA-decarboxylase inhibitor (e.g., carbidopa), which is also accompanied by adverse side effects.

Newer drug refinements and developments include direct-acting dopamine agonists, slow-release L-DOPA formulations, inhibitors of the dopamine degrading enzymes catechol-O-methyltransferase (COMT) and monoamine oxidase B (MAO-B), and dopamine transport blockers. These treatments enhance central dopaminergic neurotransmission during the early stages of Parkinson's disease, ameliorate symptoms associated with Parkinson's disease, and temporarily improve the quality of life. However, despite improvements in the use of L-DOPA for treating Parkinson's disease, the benefits accorded by these dopaminergic therapies are temporary, and their efficacy declines with disease progression. In addition, these treatments are accompanied by severe adverse motor and mental effects, most notably dyskinesias at peak dose and “on-off” fluctuations in drug effectiveness (Poewe and Granata, in Movement Disorders. Neurological Principles and Practice (Watts and Koller [eds]) McGraw-Hill, N.Y. [1997]; and Marsden and Parkes, Lancet 1:345-349 [1977]). No drug treatments are currently available that lessen the progressive pace of nigrostriatal degeneration, postpone the onset of illness, or that substantively slow disability (Shoulson, supra). Thus, currently used therapies for treating Parkinson's disease are primarily directed at symptomatic relief, are often associated with debilitating side-effects, lose efficacy over time, are difficult to administer to the brain, and provide poor long term management of the disease.

Similarly, currently used therapies for Alzheimer's disease, Huntington's disease and amyotrophic lateral sclerosis suffer the same limitations associated with Parkinson's disease therapies described above (See e.g., Sramek et al., Drugs & Aging 14:359-373 [1999]; Mayeux and Sano, N. Eng. J. Med., 341:1670-1679 [1999]; Eisen and Weber, Drugs & Aging, 14:173-196 [1999]; Borasio et al., Neurology 51:583-586 [1998]; Riviere et al., Arch. Neurol., 55:526-528 [1998]; Rosas et al., Movement Dis., 14:326-330 [1999]; Kopyov et al., Exp. Neurol., 149:97-108 [1998]; and Haque et al., Mol. Med. Today 3:175-183 [1997]). These treatments are primarily directed at symptomatic relief, are often associated with severe side-effects, lose efficacy over time, are difficult to administer to the central nervous system, and provide poor long term disease management.

Thus, new methods for the treatment of neurodegenerative diseases, including but not limited to Parkinson's disease, Alzheimer's disease, Huntington's disease and amyotrophic lateral sclerosis, that are effective and convenient, but lacking in significant side effects are needed.

Apoptosis (i.e., programmed cell death), which plays a fundamental role in the development of the nervous system (Oppenheim, Ann. Rev. Neurosci., 14: 453-501 [1991]), appears to underlie many neurodegenerative diseases and influences many of the treatment methods and considerations for treatment of neurodegenerative diseases.

For example, Alzheimer's disease (AD) is characterized by neuronal death, βamyloid (Aβ) deposition, neurofibrillary pathology, dystrophic neurites and loss of synaptic terminals (Yankner, 1996; Pettmann and Henderson, 1998; Selkoe, 1999). Aβ-induced neuronal apoptosis has been demonstrated in sympathetic neurons, cortical and hippocampal cultures and PC12 cells (Troy et al., 2001; Bozyczko-Coyne et al., 2001; Morishima et al., 2001). The stress-activated protein kinase/c-Jun N-terminal kinase (SAPK/JNK) pathway is directly associated with apoptotic events resulting from Aβ induction, including increased JNK activity, c-Jun phosphorylation, and expression of immediate early genes, c-Jun and c-Fos, as well as activation of caspase and calpain-mediated proteolysis (Estus et al., 1997; Bozyczko-Coyne et al., 2001; Troy et al., 2001). In the human hippocampus, AD pathogenesis is correlated with activation of SAPK family members, p38 and JNK, along with increased caspase3 expression (Anderson et al., 1996; Hensley et al., 1999; Gervais et al., 1999; Zhu et al., 2001).

Apoptotic pathways are mediated by cell death receptors sharing regions of homology called death domains (DD). Transduction of extracellular signals occurs in part through DD-containing proteins, such as p55 tumor necrosis factor receptor I (TNFRI), FAS receptor (FASR), TNFR associated death domain protein (TRADD) and the FASR-associated death domain protein (FADD) (Wallach et al., 1999). Recently, JNK-mediated cell death in cerebellar granule neurons and Aβ-induced apoptosis of cortical neurons were shown to promote c-Jun-dependent activation of the Fas ligand-Fas signaling pathway (Le-Niculescu et al., 1999; Morishima et al., 2001).

DENN/MADD (Differentially Expressed in Normal versus Neoplastic/MAPK Activating Death Domain containing), also referred to herein as DENN, is a TNFRI and JNK3 interacting protein. It has been shown that, for non-neuronal cells, DENN is involved in mitogen-activated protein kinase (MAPK) activation and Rab G-protein regulation (Chow and Lee, 1996; Schievella et al., 1997; Wada et al., 1997; Zhang et al., 1998; Murakami-Mori et al., 1999; Brinkman et al., 1999). However, heretofore, no studies exist that specifically address the role of DENN in neuronal apoptosis.

In the CNS, DENN, which shares 95% homology with Rab3 rat guanine nucleotide exchange protein (GEP) and 37% homology with Caenorhabditis elegans Aex3, may function under normal conditions in synaptic transmission, vesicular trafficking and neurotransmitter release (Iwasaki et al., 1997; Wada et al., 1997). Overexpression of Rat GEP has demonstrated Ca2+ dependent transmitter release and synaptic terminal localization (Oishi et al., 1998).

Acute environmental stresses on neurons activate the SAPK members, JNK and p38 (Xia et al., 1995). DENN is expressed in all neurons, while JNK3 is most strongly expressed in CA1 pyramidal neurons of the human hippocampus and is differentially distributed in neurons selectively vulnerable to oxidant stress (Mohit et al., 1995; Zhang et al., 1998). Sustained JNK activity and c-Jun phosphorylation are implicated in neuronal stress responses in neurodegenerative disorders (Herdegen et al., 1998).

This invention provides novel treatment methods for treating neurodegenerative diseases based on new understanding of apoptotic phenomena that underlie neural diseases. In particular, the invention is based on the role of DENN in neuronal apoptotic events, such as increased JNK activation and c-Jun phosphorylation, observed in neural disease.

### SUMMARY OF THE INVENTION

The invention describes methods for treating neurodegenerative diseases by modulating the expression of DENN in neuronal cells. It has been observed that neurodegenerative disease states are characterized by abnormal expression of DENN. The overexpression of DENN induces cell death in neuronal cells. However, reduced expression of DENN also characterizes neural tissue affected by neurodegenerative disease.

In one embodiment, the invention provides methods for treating neurodegenerative diseases by inhibiting the interaction of DENN-MADD (Differentially Expressed in Normal versus Neoplastic/MAPK Activating Death Domain containing) protein, also referred to herein as DENN, with c-Jun N-terminal kinases (JNKs). The invention further describes methods for treating neurodegenerative diseases by inhibiting the interaction of DENN-MADD with the p55 tumor necrosis factor receptor I (TNFRI).

Compositions that can be used with the methods of the invention comprise antisense oligonucleotides of the DENN-MADD gene, which include those with sequences substantially equivalent to any of the sequences SEQ ID NO: 1 to SEQ ID NO: 5, including fragments thereof. Oligonucleotides of the present invention also include, but are not limited to, oligonucleotides complementary to the nucleotide sequence of each of SEQ ID NO: 1-5.

Alternative compositions used in the methods of the invention comprise chemical derivatives, including synthetic derivatives, of the indolocarbazole commonly known as K-252a.

Further, the treatment methods of the invention may use compositions that comprise pyridyl imidazoles, defined below.

In alternative embodiment, the treatment methods of the invention are used in conjunction with another method of treating a neurological disorder.

The compositions used in the methods of the invention may further comprise a pharmaceutically acceptable carrier. Preferably, the composition is administered orally, transdermally, intravenously, intrasynovially, intramnuscularly, intraocularly, intranasally, intrathecally, or topically. In alternative embodiment, the composition of the invention is administered in conjunction with another method of treating a neurological disorder.

The neurological disorder may be caused by oxidative stress response in neuronal tissue. It may be caused by the activation of a neuron specific, stress-activated protein kinase, such as c-Jun amino-terminal kinase 3. Specifically, the neurological disorder is a disorder selected from dementia, dementia of the Alzheimer's type, bipolar disorders, mood disorder with depressive features, mood disorder with major depressive-like episode, mood disorder with manic features, mood disorder with mixed features, substance-induced mood disorder and mood disorder not otherwise specified (NOS), panic disorder without agoraphobia, panic disorder with agoraphobia, agorathobia without history of panic disorder, social phobia, postraumatic stress disorder, acute stress disorder, substance-induced anxiety disorder and anxiety disorder not otherwise specified (NOS), dyskinesias and behavioral manifestations of mental retardation, conduct disorder and autistic disorder. The dementia may be dementia selected from the group consisting of vascular dementia, dementia due to HIV disease, dementia due to head trauma, dementia due to Parkinson's disease, dementia due to Huntington's disease, dementia due to Pick's disease, dementia due to Creutzfeldt-Jakob disease, substance-induced persisting dementia, dementia due to multiple etiologies and dementia not otherwise specified (NOS). Alternatively, the dementia is dementia of the Alzheimer's type, which may be selected from the group consisting of dementia of the Alzheimer's type with early onset uncomplicated, dementia of the Alzheimer's type with early onset with delusions, dementia of the Alzheimer's type with early onset with depressed mood, dementia of the Alzheimer's type with late onset uncomplicated, dementia of the Alzheimer's type with late onset with delusions and dementia of the Alzheimer's type with late onset with depressed mood.

Preferably, the composition is administered in a targeted drug delivery system, for example, a targeted drug delivery system such as a liposome coated with an antibody that specifically targets neuronal tissue.

The methods of the present invention further relate to the methods and kits for detecting the presence of the products of the interaction between DENN-MADD and one or more JNKs in a sample, or the products of the interaction between DENN-MADD and TNFRI. Such methods and kits can, for example, be utilized as a prognostic indicator of stroke or Alzheimer's disease, or Parkinson's disease or amyotrophic lateral sclerosis (Lou Gehrig's disease).

### BRIEF DESCRIPTION OF THE DRAWINGS

FIG. 1 illustrates the results of experiments that show that overexpression of DENN specifically induces cell death in neuronal cells. A) Schematic diagram of DENN in signal transduction pathways include death domain proteins and JNK. Both pathways lead to apoptosis. DENN binds to JNK3 and TNFR1 through distinct separate domains. JNK3, activated by upstream kinases, including MKK4 and MEKK1, phosphorylates downstream targets including c-Jun. TNFR1 binds death domain proteins such as TRADD and induces apoptosis. B) Plasmids encoding N-terminal GFP fusion proteins of DENN, TRADD, FADD, TNFR1 and dominant negative FADD (DNFADD) were transiently transfected with FLAG-tagged JNK3 into two neuronal cell lines, (NSC 19, N₂A) and non-neuronal cultures (293 and COS7). Forty-eight hours post-transfection, cells were collected, stained with Viaprobe (7-AAD) and processed by FACS analysis. The percentage of cell death represents the number of GFP and 7-AAD positive cells relative to total GFP positive cells sorted from 10,000 events. Note neurospecificity of DENN. Data are mean± SEM (bars) values of three independent experiments.

FIG. 2 illustrates the results of experiments that show that expression of the C-terminal death domain region of DENN is sufficient for apoptosis. A) Domain structure of full length DENN includes a putative nuclear localization signal (NLS), leucine zipper region (LZ), JNK3 binding domain (JBD) and death domain (DD). Below, schematic maps of DENN mutants fused to the C-terminus of GFP. DENN-DD contains the death domain only, and DENN-CT includes the DD and C-terminal region of DENN. DENN-ΔCT is truncated at residue 1292 and lacks both the DD and C-terminus. DENN-JBD lacks both the regions spanning the putative DD and NLS regions. Amino acids included in these constructs are shown at the right. B) Western blot of GFP fusions of DENN/MADD and DENN/MADD mutants expressed in NSC 19 cells after transfection. For each sample, 50 μg of cell extract were subjected to 10% SDS-PAGE and detected by anti-GFP monoclonal antibody, labeled with horseradish peroxidase-conjugated anti-mouse IgG, and detected by chemiluminescence. Protein size markers (kD) are at right. C) Fluorescence microscopy of NSC19 transfectants of GFP and GFP fusions of DENN, DENN-CT, DENN-ΔCT and DENN-JBD. Cell rounding occurs prior to cell death as seen in DENN-CT. D) Induction of apoptosis by DENN mutants assessed by flow cytometry. At 24 and 48 h post-transfection, NSC19 cells were collected and phycoerythrin conjugated-annexin V (PE-annexin V) was added to detect apoptotic cells percentages of apoptotic cells is the number of cells positive for GFP and annexin V relative to total GFP expressing cells. Data are mean± SEM of triplicate cultures.

FIG. 3 illustrates the results of experiments that show that DENN co-localizes with JNK in vivo. Full length DENN and FLAG-JNK3 were overexpressed in NSC19 cells and incubated for 24 h. Cells were fixed in 4% paraformaldehyde (PFA) and stained with antibodies specific for DENN and the FLAG epitope of the transfected JNK3. For confocal microscopy, DENN was labeled with Texas Red (A), and FLAG-JNK3 using the FITC filter (B). Merged image is in (C). DENN and JNK3 co-localize in the cytoplasm in distinct aggregates (yellow); (lower cell, arrow). Some cells still maintain processes (upper cell) or begin to round (lower cell). Endogenous expression of DENN (D) and phospho-JNK (E) in N₂A cells with merged image (F) demonstrates co-localization with phospho-JNK in cytoplasm and nucleus of select neurons. COS7 cells transfected with DENN and FLAG-JNK3 show co-localization in the cytoplasm and perinuclear region (arrow) (G). Primary mouse hippocampal neurons grown for 7 days, fixed in 4% PFA and stained with antibodies specific for MAP2 (green) and DENN (red) (H) or phospho-JNK (green) and DENN (red) (I) show significant expression of DENN in hippocampal neuron and have perinuclear co-localization of endogenous DENN and phospho-JNK (arrows). Scale bars are 10 μm in A through F and 20 μm in G-I.

FIG. 4 illustrates the results of experiments that show that DENN overexpression enhances Aβ-induced apoptosis. A) NSC19 cell clones overexpressing GFP, and GFP fusions of DENN, CT or JBD, were exposed to 20 μM Aβ₍₁₋₄₀₎ for 1, 2 or 3 days. Cell death was monitored by FACS analysis of GFP and propidium iodide (PI) positive cells. DENN and CT transfection alone induce cell death in the absence of Aβ, but is increased with Aβ exposure. GFP and JBD transfected cells yield significantly less cell death, but also increases in response to Aβ exposure. B) Aβ peptide promotes increased apoptosis in cells overexpressing DENN. Cell clones overexpressing GFP or DENN were treated with 0.5% BSA, UV-C irradiation for 5 min or 20 μM Aβ peptides: Aβ(40-1), Aβ(25-35), Aβ(1-40) or Aβ(1-42), for 24 h. Cell were stained with PE-annexin V and analyzed by FACS. Data are mean± SEM of triplicate cultures. Increases in apoptosis were seen with Aβ peptide and UV-C exposure, but UV irradiation and reverse peptide Aβ(40-1) exposure showed minimal differences between GFP alone and DENN. C) The C-terminal domain of DENN is critical for Aβ-induced cell death. GFP-tagged deletion mutants of DENN were expressed in NSC19 cells and exposed to 20 μM Aβ(1-40) for 48 h. Aβ induced cell death compared to GFP/−Aβ and GFP/+Aβ, p<0.001. DENN, DENN-CT and DENN-DD mutants demonstrated significant increases in cell death by FACS analysis of GFP and PI positive cells, ANOVA, p<0.01. D) N₂A cells overexpressing GFP or DENN were treated with 20 μM Aβ₍₁₋₄₀₎ for indicated times and genomic DNA extracted. Equal amounts of DNA were run on 1.5% agarose gel clectrophoresis and stained with ethidium bromide. Arrows on right indicate nucleotide base pairs. Progressive nuclear fragmentation was seen over time and detectable by 12 h Aβ exposure in DENN expressing cells.

FIG. 5 illustrates the results of experiments that show that DENN overexpression activates the JNK pathway. A) DENN induces JNK phosphorylation. NSC19 cell clones expressing DENN, DENN-CT, DENN-JBD, GFP (Control) and ΔMEKK1 were exposed to 25 μM Aβ₍₁₋₄₂₎ for 24 h. Cell extracts (50 μg) were probed with antibodies to phosphorylated JNK (Thr183/Tyr185), phosphorylated ERK, phosphorylated p38, and total JNK. DENN, DENN-JBD, DENN-CT and ΔMEKK1 show p46 and p54 phospho-JNK reactive bands compared to GFP control with Aβ increasing the p54 band in DENN and JBD transfectants. Total JNK levels remain constant. ERK activation is induced ΔMEKK1 and DENN transfectants regardless of Aβ and p38 shows no activation. B) NSC19 cell clones expressing GFP (vector), DENN, DENN-CT, DENN-JBD and ΔMEKK1 were exposed to 25 μM Aβ(1-42) for 4 h. Cell extracts (50 μg) were immunoblotted with an antibody to phosphorylated c-Jun (Ser73). DENN, DENN-JBD and ΔMEKK1 all show increased c-Jun phosphorylation, while GFP and DENN-CT show modest levels. Aβ only minimally increases c-Jun phosphorylation in DENN and JBD. Total c-Jun expression remained unchanged in lower panel. C) SB203580 attenuates DENN-induced apoptosis. NSC19 cells overexpressing GFP, DENN, DENN-CT and DENN-JBD were exposed to 0.1% DMSO (control), 10 μM SB203580, 10 , M Aβ(1-40) or 10 μM SB203580 with 10 μM Aβ(1-40) for 24 h. Cells were collected and incubated with PE-annexin V and analyzed by FACS for GFP and annexin V positive cells. Data are averages±SEM of two independent experiments. Enhanced Aβ-induced apoptosis in DENN and DENN-CT cultures were decreased with addition of SB203580, while DENN-JBD demonstrated a slight increase in apoptosis. D) N₂A cultures overexpressing GFP or DENN were exposed to 25 μM Aβ(1-42) or 25 μM Aβ(1-42) with 1 μM CEP11004 for 24 h. JNK was immunoprecipitated from cell extracts and incubated with GST-cJun(1-79) substrate in an in vitro kinase assay. Reaction products were immunoblotted with antibody specific for phosphorylated c-Jun. Representative gel of three independent experiments is shown. Aβ treated DENN-transfectant markedly activates c-Jun compared to vector only transfectants. CEP11004 inhibits Aβ-induced c-Jun phosphorylation in both GFP and DENN cell cultures, however increased c-Jun phosphorylation in DENN overexpressing cells is more effectively inhibited.

FIG. 6 illustrates the results of experiments that show that endogenous DENN is reduced in neuronal cells when stressed with Aβ. A) NSC19 and N₂A cells were exposed to 0.1% BSA (Control) or 20 μM fibrillar Aβ₍₁₋₄₀₎ for 48 h. Cell extracts (100 μg) were immunoblotted with DENN antibody and reprobed with actin antibody. DENN reactive bands appear at ˜200 kD and ˜140 kD. DENN protein expression is higher in NSC19 relative to N₂A cells. Aβ exposure dramatically reduces DENN protein expression. Similar results were obtained in three independent experiments. B) Total RNA from Control and Aβ exposed N₂A cultures was extracted and RT-PCR using either DENN or actin specific primers was performed. RNA levels of DENN are reduced in Aβ exposed cultures, while actin levels are comparable. Results are representative of three independent experiments. C) Primary rat hippocampal neurons were exposed to 25 μM Aβ₍₁₋₄₂₎ for 24 h, fixed in 4% PFA and stained with antibodies specific for DENN, phospho-JNK (P-JNK) and counterstained with Hoechst 33342 for nuclei. Merged image shows overlap of DENN (green) and phospho-JNK (red) with decreased expression of DENN and nuclear translocation of phopho-JNK in Aβ exposed cultures.

FIG. 7 illustrates the results of experiments that show that AD-affected human hippocampus exhibits reduced DENN expression. A) RT-PCR of total RNA pooled from two age-matched control and AD cases were amplified using primers specific for DENN or β-actin. RNA expression of DENN is significantly reduced in AD hippocampus, while actin levels are constant. B) Western blotting of four age-matched control and AD cases. Homogenates of hippocampi were prepared and 200 μg per lane was analyzed on 10% SDS-PAGE, transferred to nitrocellulose and probed with antibodies specific for DENN, TRADD, FADD, TNFR1 and JNK3. DENN exhibits a specific band at 140 kD that is reduced in AD hippocampal homogenates. TRADD shows increased protein expression in AD while FADD, TNFR1 and JNK3 are relatively constant in both control and AD tissues. C) Immunohistochemical (immunoperoxidase) localization of DENN in control and AD hippocampus. Immunostaining of control area CA4 shows strong cytoplasmic staining, while in AD area CA4 has less staining and CA1 is dramatically reduced, with nuclear localization in some neurons (arrowhead). Bar=20 μm.

FIG. 8 illustrates the results of experiments that show that reduced DENN expression in hippocampus is co-localized with AD vulnerable regions and altered activated MAPK/SAPK expression. CA1 regions of human hippocampus were stained with antibodies specific to DENN and AD markers or activated MAPKs. A) DENN staining in representative Control case. Control shows cytoplasmic localization of DENN in pyramidal neurons. AD cases were immunostained with antibodies specific for DENN (labeled with Alexa488, green) and monoclonal antibodies which recognize granulovacuolar degeneration (3A4) (B) and phosphorylated tau (AT8); (Alexa562, red) (C). Nuclei are labeled with Hoechst 33342. Arrows indicate either GVD or tau localization. AD shows diminished DENN expression in regions with significant GVD or phosphorylated tau. Staining of AD hippocampus with (D) phospho-JNK (JNK-P) or (E) phospho-ERK (ERK-P) shows nuclear overlap (arrows) and absence of stain, respectively. As a control in AD, phosphorylated p38 (p38-P) is highly immunoreactive in nuclei of neurons in tau-affected regions (F). (G,H) Lower magnification of DENN and phospho-JNK immunoreactivity in CA1 and CA4 regions of AD hippocampus. Absence of DENN stain in CA1 and reduced expression in CA4 and dentate gyrus, respectively, is seen with significant phospho-JNK stain.

FIG. 9 illustrates the results of experiments that show that anti-sense inhibition of DENN expression leads to cell death in primary neurons. (A) Control-AS and DENN-AS were transfected into 8 DIV mouse hippocampal neurons using oligonucleotides against the DENN JBD region (DENN-AS) or a scrambled sequence (Control-AS). Neurons were exposed to AS for 2 d, fixed in 4% PFA and stained with antibodies specific to MAP2 and DENN. The merged image indicates greatly diminished expression of DENN in the DENN-AS treated cultures. (B) Control or DENN-AS treated cultures were exposed to 25 μM Aβ₍₁₋₄₂₎ for 24 h and stained with DAPI (blue) for nuclei and TUNEL (green) for ssDNA as a measure of cell death. TUNEL positive cells are increased in DENN-AS treated cultures relative to Control. (C) The percentage of TUNEL positive cells from hippocampal cultures treated with either Control or DENN-AS alone, with 25 μM Aβ₍₁₋₄₂₎ for 24 h, or with 25 μM Aβ₍₁₋₄₂₎+1 μM CEP11004 for 24 h. DENN-AS treated cultures show increased TUNEL positive cells, while CEP1004 inhibits effect of Aβ-induced cell death.

### DETAILED DESCRIPTION

This invention is based on a novel understanding of the pathways implicated in neurodegenerative diseases. In particular, it has been discovered that abnormal expression of DENN and its interaction with other proteins. such as JNKs and TNFRI, in neuronal apoptotic pathways plays a central role in neural disease.

In Alzheimer's disease (AD), gradual accumulation of β-amyloid (Aβ) results in a multi-step process of impaired function, induction of protective mechanisms and ultimately cell death: Signal transduction mechanisms mediating Aβ-induced neuronal death are not yet fully understood.

This invention is based on the novel understanding of the role of DENN/MADD in regulating apoptosis mediated by Aβ involving activation of the JNK pathway. As the examples below show, DENN/MADD overexpression induced cell death in neuronal cells relative to non-neuronal cells comparable to DD containing proteins: TNFRI associated, death domain protein (TRADD) and FAS receptor associated death domain protein (FADD). The examples also show that C-terminal domain of DENN/MADD, spanning a region with homology to the DD of TNFRI and TRADD, is essential for maximum cytotoxicity. The examples also show that DENN/MADD co-localized with JNK3 in the cytoplasm of transfected NSC19 cells and with activated JNK in the perinuclear cytoplasm and neuritic processes of murine and rat hippocampal primary cultures. Further, the examples show that overexpressed DENN/MADD increased neuronal vulnerability to Aβ-induced cell death in neuroblastoma cells and JNK activation and c-Jun phosphorylation were increased in cells overexpressing DENN/MADD, while inhibitors of JNK activation attenuated DENN/MADD-induced cell death and c-Jun phosphorylation, respectively.

Conversely, Aβ stress decreased endogenous DENN/MADD expression in neuronal cultures as assessed by Western blot, immunocytochemistry and RT-PCR. Nuclear localization of JNK occurred with decreased DENN/MADD expression and correlated with increased cell death in Aβ-exposed neuronal cultures and AD neuropathology in human hippocampus. Western blotting and RT-PCR of AD-affected tissue and antisense treatment of primary rat hippocampal cultures further confirmed reduction in DENN/MADD expression. In CA1 area of the AD hippocampus, tau pathology and granulovacuolar degeneration (GVD) DENN/MADD was also decreased. In normal hippocampus, DENN co-localized with activated JNK and ERK in the neuronal cytoplasm, while in AD, activated MAPKs and DENN/MADD were reduced in pyramidal neurons. The examples show that DENN is a key regulator of MAPK/SAPK pathways in the etiology of AD pathogenesis.

### GENERAL DEFINITIONS

The term “nucleotide sequence” refers to a heteropolymer of nucleotides or the sequence of nucleotides. One of skill in the art will readily discern from contextual cues which of the two definitions is appropriate. The terms “nucleic acid” and “polynucleotide” are also used interchangeably herein to refer to a heteropolymer of nucleotides. Generally, nucleic acid segments provided by this invention may be assembled from fragments of the genome and short oligonucleotide linkers, or from a series of oligonucleotides, or from individual nucleotides, to provide a synthetic nucleic acid which is capable of being expressed in a recombinant transcriptional unit comprising regulatory elements derived from a microbial or viral operon, or a eukaryotic gene.

The term “probes” includes naturally occurring or recombinant or chemically synthesized single- or double-stranded nucleic acids. They may be labeled by nick translation, Klenow fill-in reaction, PCR or other methods well known in the art. Probes of the present invention, their preparation and/or labeling are elaborated in Sambrook, J. et al., 1989. Molecular Cloning: A Laboratory Manual, Cold Spring Harbor, N.Y.; or Ausubel, F. et al., 1989, Current Protocols in Molecular Biology, John Wiley & Sons, New York, both of which are incorporated herein by reference in their entirety.

The term “recombinant expression system” means host cells which have stably integrated a recombinant transcriptional unit into chromosomal DNA or carry the recombinant transcriptional unit extrachromosomally. Recombinant expression systems as defined herein will express heterologous polypeptides or proteins upon induction of the regulatory elements linked to the DNA segment or synthetic gene to be expressed. This term also means host cells which have stably integrated a recombinant genetic element or elements having a regulatory role in gene expression, for example, promoters or enhancers. Recombinant expression systems as defined herein will express polypeptides or proteins endogenous to the cell upon induction of the regulatory elements linked to the endogenous DNA segment or gene to be expressed. The cells can be prokaryotic or eukaryotic.

The term “active” refers to those forms of the polypeptide which retain the biologic and/or immunologic activities of any naturally occurring polypeptide.

The term “naturally occurring polypeptide” refers to polypeptides produced by cells that have not been genetically engineered and specifically contemplates various polypeptides arising from post-translational modifications of the polypeptide including, but not limited to, acetylation, carboxylation, glycosylation, phosphorylation, lipidation and acylation.

The term “derivative” refers to polypeptides chemically modified by such techniques as ubiquitination, labeling (e.g., with radionuclides or various enzymes), pegylation (derivatization with polyethylene glycol) and insertion or substitution by chemical synthesis of amino acids such as ornithine, which do not normally occur in human proteins.

As used herein, the term “pyridyl imidazole” encompasses compounds comprising a pyridine and an imidazole ring, including compounds comprising a substituted pyridine and a substituted imidazole. In preferred embodiments, the pyridyl imidazoles of the present invention encompasses compounds comprising a pyridine substituted with an imidazole ring, and derivatives thereof. Examples of pyridyl imidazole derivatives for use in the methods of the present invention include, but are not limited to PD 169316, isomeric PD 169316, RWJ 67657, SB 203580, SB 202190, SB 220025 (the chemical structures of these compounds are provided below), as well as compounds described in U.S. Pat. No.: 5,656,644 to Adams et al. (hereby incorporated by reference in its entirety) and compounds described in U.S. Pat. No. 6,214,830 (hereby incorporated by reference in its entirety) and isomers and derivatives thereof.

JNKs, Jun N-terminal kinases, are MAP kinases. JNKs have been implicated as key mediators of a variety of cellular responses and pathologies. JNKs can be activated by environmental stress, such as radiation, heat shock, osmotic shock, or growth factor withdrawal as well as by pro-inflammatory cytokines. Several studies have demonstrated a role for JNK activation in apoptosis induced by a number of stimuli in several cell types. This invention is partly based on the novel discovery that JNKs are activated by their interaction with DENN.

Several JNKs are known. For example, JNK1 and JNK2 are responsible for the phosphorylation of specific sites (Serine 63 and Serine 73) on the amino terminal portion of c-Jun. Phosphorylation of these sites potentiates the ability of AP-1 to activate transcription (Binetruy et al., Nature, 1991, 351, 122; Smeal et al., Nature, 1991, 354, 494). Besides JNK1 and JNK2, other JNK family members have been described, including JNK3 (Gupta et al., EMBO J., 1996, 15, 2760). The term “JNK protein” as used herein shall mean a member of the JNK family of kinases, including but not limited to JNK1, JNK2 and JNK3, their isoforms (Gupta et al., EMBO J., 1996, 15, 2760) and other members of the JNK family of proteins whether they function as Jun N-terminal kinases per se (that is, phosphorylate Jun at a specific amino terminally located position) or not.

In addition to the initially discovered JNK1 (Derijard et al., Cell, 1994, 76, 1025), cDNAs encoding related isoforms of JNK1 have been cloned and their nucleotide sequences determined (Gupta et al., EMBO Journal, 1996, 15, 2760). In addition to JNK1-α1 (GenBank accession No. L26318, locus name “HUMJNK1 ”), which encodes a polypeptide having an amino acid sequence identical to that of JNK1, the additional isoforms include JNK1-α2 (GenBank accession No. U34822, locus name “HSU34822”), JNK1-α1 (GenBank accession No. U35004, locus name “HSU35004”) and JNK1-β2 (GenBank accession No. U35005, locus name “HSU35005”).

In addition to the initially discovered JNK2 (Sluss et al., Mol. Cel. Biol., 1994, 14, 8376; Kallunki et al., Genes & Development, 1994, 8, 2996; GenBank accession No. HSU39759, locus name “U09759”), cDNAs encoding related isoforms cf JNK2 have been cloned and their nucleotide sequences determined (Gupta et al., EMBO Journal, 1996, 15, 2760). In addition to JNK2-α2 (GenBank accession No. L31951, locus name “HUMJN2”), which encodes a polypeptide having an amino acid sequence identical to that of JNK2, the additional isoforms include JNK2-α1 (GenBank accession No. U34821, locus name “HSU34821”), JNK2-β1 (GenBank accession No. U35002, locus name “HSU35002”) and JNK2-β2 (GenBank accession No. U35003, locus name “HSU35003”).

Two isoforms of JNK3 have been described: JNK3-α1 (GenBank accession No. U34820, locus name “HSU34820”) and JNK3-α2 (GenBank accession No. U34819, locus name “HSU34819”).

As used herein, “substantially equivalent” can refer both to nucleotide and amino acid sequences, for example a mutant sequence, that varies from a reference sequence by one or more substitutions, deletions, or additions, the net effect of which does not result in an adverse functional dissimilarity between the reference and subject sequences. Typically, such a substantially equivalent sequence varies from one of those listed herein by no more than about 2% (i.e., the number of individual residue substitutions, additions, and/or deletions in a substantially equivalent sequence, as compared to the corresponding reference sequence, divided by the total number of residues in the substantially equivalent sequence is about 0.02 or less). Such a sequence is said to have 98% sequence identity to the listed sequence. hi one embodiment, a substantially equivalent, e.g., mutant, sequence of the invention varies from a listed sequence by no more than 2% (98% sequence identity); in a variation of this embodiment, by no more than 0.5% (99.5% sequence identity); and in a further variation of this embodiment, by no more than 0.1% (99.9% sequence identity). Substantially equivalent, e.g., mutant, amino acid sequences according to the invention generally have at least 98% sequence identity with a listed amino acid sequence, whereas substantially equivalent nucleotide sequence of the invention can have lower percent sequence identities, taking into account, for example, the redundancy or degeneracy of the genetic code. For the purposes of determining equivalence, truncation of the mature sequence (e.g., via a mutation which creates a spurious stop codon) should be disregarded.

The term “purified” as used herein denotes that the indicated nucleic acid or polypeptide is present in the substantial absence of other biological macromolecules, e.g., polynucleotides, proteins, and the like. In one embodiment, the polynucleotide or polypeptide is purified such that it constitutes at least 95% by weight, more preferably at least 99.8% by weight, of the indicated biological macromolecules present (but water, buffers, and other small molecules, especially molecules having a molecular weight of less than 1000 daltons, can be present).

The term “isolated” as used herein refers to a nucleic acid or polypeptide separated from at least one other component (e.g., nucleic acid or polypeptide) present with the nucleic acid or polypeptide in its natural source. In one embodiment, the nucleic acid or polypeptide is found in the presence of (if anything) only a solvent, buffer, ion, or other component normally present in a solution of the same. The terms “isolated” and “purified” do not encompass nucleic acids or polypeptides present in their natural source.

The term “infection” refers to the introduction of nucleic acids into a suitable host cell by use of a virus or viral vector.

The term “transformation” means introducing DNA into a suitable host cell so that the DNA is replicable, either as an extrachromosomal element, or by chromosomal integration.

The term “transfection” refers to the taking up of an expression vector by a suitable host cell, whether or not any coding sequences are in fact expressed.

Each of the above terms is meant to encompasses all that is described for each, unless the context dictates otherwise.

### Indolocarbazoles

The methods of the invention comprise administering compositions for modulating the expression of DENN, and/or inhibiting the interaction of DENN with JNKs and/or TNFRI. Amongst such compositions are compositions comprising indolocarbazoles. An example indolocarbazole is the indolocarbazole referred to as CEP-11004 from Cephalon, Inc., which has been shown to be efficacious in the methods of the invention.

CEP-11004 is a semisynthetic derivative of the fermentation product K-252a, an indolocarbazole alkaloid. The microbial-derived material referred to as “K-252a” is a unique compound which has gained significant attention over the past several years due to the variety of functional activities which it possesses. K-252a is an indolocarbazole alkaloid that was originally isolated from a Nocordiosis sp. culture (Kase, H et al. 39 J. Antibiotics 1059, 1986). K-252a is an inhibitor of several enzymes, including protein kinase C (“PKC”) and trk tyrosine kinase. The reported functional activities of K-252a are numerous and diverse: tumor inhibition (U.S. Pat. Nos. 4,877,776 and 5,063,330; European Publication 238,011 in the name of Nomato); anti-insecticidal activity (U.S. Pat. No. 4,735,939); inhibition of inflammation (U.S. Pat. No. 4,816,450); treatment of diseases associated with neuronal cells (WIPO Publication WO 94/02488, published Feb. 3, 1994 in the names of Cephalon, Inc. and Kyowa Hakko Kogyo Co., Ltd.).

The reported indolocarbazoles share several common attributes: in particular, each comprises three five member rings which all include a nitrogen moiety; staurosporine (derived from Streptomyces sp.) and K-252a (derived from Nocordiosis sp.) each further comprise a sugar moiety linked via two N-glycosidic bonds. Both K-252a and staurosporine have been extensively studied with respect to their utility as therapeutic agents. The indolocarbazoles are generally lypophilic which allows for their comparative ease in crossing biological membranes, and, unlike proteinaceous materials, they manifest a longer in vivo half life.

Although K-252a is normally derived from culture media via a fermentation process, the total synthesis of the natural (+) isomer and the unnatural (−) isomer, in which the three chiral carbons of the sugar have the opposite configurations, has been achieved (See Wood et al., J. Am. Chem. Soc. 117: 10413, 1995, and WIPO Publication WO 97/07081).

In addition to the indolocarbazole alkaloids represented by K-252a and staurosporine, synthetic small organic molecules which are biologically active and known as fused pyrrolocarbazoles are contemplated to be used with the methods of the invention. Chemical formulas and methods for preparation of these compounds are provided in U.S. Pat. Nos. 5,475,110; 5,591,855; 5,594,009; 5,705,511; and 5,616,724, each of which is incorporated in its entirety by reference herein.

Fused isoindolones which are non-indole-containing molecules that can be chemically synthesized de novo are also contemplated to be used with the methods of the invention. See U.S. Pat. No. 5,808,060 and WIPO Publication WO 97/21677, each of which is incorporated in its entirety by reference herein, for chemical formulas and methods of synthesis for these compounds. Certain bis-indolylmaleimide macrocyclic derivatives are also contemplated to be used with the methods of the invention. See U.S. Pat. Nos. 5,710,145; 5,672,618; 5,552,396 and 5,545,636, each of which is incorporated in its entirety by reference herein, for chemical formulas and methods of synthesis for these compounds. Sugar derivatives of indolopyrrolocarbazoles also have been reported (see WIPO Publication WO98/07433). There remains a need for novel pyrrolocarbazole and isoindolone derivatives that possess beneficial properties. This invention is directed to this, as well as other, important ends.

Other functional derivatives of K-252a may be prepared chemical synthesis using methods known to those skilled in the art and the use of such functional derivatives in the methods of the invention is also contemplated to be within the scope of the invention. The formulas and the chemical synthesis of various other K-252a functional derivatives is outlined in several patents, including U.S. Pat. Nos. 4,923,986, 4,877,776, 5,468, 872, 5,616,724, 5,650,407, 5,686,444, 6,093,713, 6,127,401, 6,306,849, 6,359,130, 6,399,780, each of which is incorporated in its entirety by reference herein. Additional procedures used for the preparation of such derivatives are described by Moody et al., J. Org. Chem. 57: 2105-2114 (1992); Steglich et al., Angew. Chem. Int. Ed. Engl. 19: 459-460 (1980); Nakanishi et al., J. Antibiotics 39: 1066-1071 (1986); and Japanese Patent Application No. 60-295172 (1985). Further methods are described for the compounds in Japanese Patent Application Nos. 60-295173 (1985), 62-327858 (1987), 62-327859 (1987) and 60-257652 (1985) [Meiji Seika Kaisha Ltd.].

The chemical structure of an exemplary indolocarbazole is shown below:

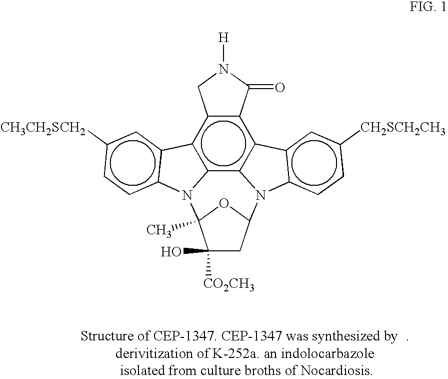

### Antisense Oligonucleotides

Oligonucleotides may comprise nucleotide sequences sufficient in identity and number to effect specific hybridization with a particular nucleic acid. Such oligonucleotides are commonly described as “antisense.” Antisense oligonucleotides are commonly used as research reagents, diagnostic aids, and therapeutic agents. It has been discovered that the gene encoding for DENN is particularly amenable to this approach.

The present invention employs oligonucleotides for use in antisense modulation of the function of DNA or messenger RNA (mRNA) encoding a protein the modulation of which is desired, and ultimately to regulate the amount of such a protein. Specifically, it has been discovered that abnormal expression of DENN causes and/or is symptomatic of neural diseases. Thus, for example, it has been discovered that overexpression of DENN leads to activation of one or more of the JNKs, triggering neurodegenerative disease. Therefore, the invention seeks to modulate the expression of DENN in subjects suffering from neurodegenerative disease by contacting the subject's cells with DENN antisense oligonucleotides.

Methods of modulating the expression of DENN comprising contacting animals with oligonucleotides specifically hybridizable with a nucleic acid encoding DENN are herein provided. These methods are believed to be useful both therapeutically and diagnostically as a consequence of the association between DENN-mediated activation of JNK and apoptosis and the central role of these phenomena in neural disease. These methods are also useful as tools, for example, in the detection and determination of the role of DENN-mediated activation of JNK in various cell functions and physiological processes and conditions associated with neural disorders, and for the diagnosis of such disorders.

Hybridization of an antisense oligonucleotide with its mRNA target interferes with the normal role of mRNA and causes a modulation of its function in cells. The functions of mRNA to be interfered with include all vital functions such as translocation of the RNA to the site for protein translation, actual translation of protein from the RNA, splicing of the RNA to yield one or more mRNA species, and possibly even independent catalytic activity which may be engaged in by the RNA. The overall effect of such interference with mRNA function is modulation of the expression of a protein, wherein “modulation” means either an increase (stimulation) or a decrease (inhibition) in the expression of the protein. In the context of the present invention, inhibition is the preferred form of modulation of gene expression.

It is preferred to target specific genes for antisense attack. “Targeting” an oligonucleotide to the associated nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a foreign nucleic acid from an infectious agent. In the present invention, the target is a cellular gene associated with hyperproliferative disorders. The targeting process also includes determination of a site or sites within this gene for the oligonucleotide interaction to occur such that the desired effect, either detection or modulation of expression of the protein, will result. Once the target site or sites have been identified, oligonucleotides are chosen which are sufficiently complementary to the target, i.e., hybridize sufficiently well and with sufficient specificity to give the desired effect. Generally, there are five regions of a gene that may be targeted for antisense modulation: the 5′ untranslated region (hereinafter, the “5′-UTR”), the translation initiation codon region (hereinafter, the “tIR”), the open reading frame (hereinafter, the “ORF”), the translation termination codon region (hereinafter, the “tTR”) and the 3′ untranslated region (hereinafter, the “3”-UTR). As is known in the art, these regions are arranged in a typical messenger RNA molecule in the following order (left to right, 5′ to 3′): 5′-UTR, tIR, ORF, tTR, 3′-UTR. As is known in the art, although some eukaryotic transcripts are directly translated, many ORFs contain one or more sequences, known as “introns,” which are excised from a transcript before it is translated; the expressed (unexcised) portions of the ORF are referred to as “exons” (Alberts et al., Molecular Biology of the Cell, 1983, Garland Publishing Inc., New York, pp. 411-415). Furthermore, because many eukaryotic ORFs are a thousand nucleotides or more in length, it is often convenient to subdivide the ORF into, e.g., the 5′ ORF region, the central ORF region, and the 3′ ORF region. In some instances, an ORF contains one or more sites that may be targeted due to some functional significance in vivo. Examples of the latter types of sites include intragenic stem-loop structures (see, e.g., U.S. Pat. No. 5,512,438) and, in unprocessed mRNA molecules, intron/exon splice sites.

Within the context of the present invention, one preferred intragenic site is the region encompassing the translation initiation codon of the open reading frame (ORF) of the gene. Because, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon.” A minority of genes have a translation initiation codon having the RNA sequence 5′-GUG, 5′-UUG or 5′-CUG, and 5′-AUA, 5′-ACG and 5′-CUG have been shown to function in vivo. Furthermore, 5′-UUU functions as a translation initiation codon in vitro (Brigstock et al., Growth Factors, 1990, 4, 45; Gelbert et al., Somat. Cell. Mol. Genet., 1990, 16, 173; Gold and Stormo, in: Escherichia coli and Salmonella typhimurium: Cellular and Molecular Biology, Vol. 2, 1987, Neidhardt et al., e's., American Society for Microbiology, Washington, D.C., p. 1303). Thus, the terms “translation initiation codon” and “start codon” can encompass many codon sequences, even though the initiator amino acid in each instance is typically methionine (in eukaryotes) or formylmethionine (prokaryotes). It is also known in the art that eukaryotic and prokaryotic genes may have two or more alternative start codons, any one of which may be preferentially utilized for translation initiation in a particular cell type or tissue, or under a particular set of conditions, in order to generate related polypeptides having different amino terminal sequences (Markussen et al., Development, 1995, 121, 3723; Gao et al., Cancer Res., 1995, 55, 743; McDermott et al., Gene, 1992, 117, 193; Perri et al., J. Biol. Chem., 1991, 266, 12536; French et al., J. Virol., 1989, 63, 3270; Pushpa-Rekha et al., J. Biol. Chem., 1995, 270, 26993; Monaco et al., J. Biol. Chem., 1994, 269, 347; DeVirgilo et al., Yeast, 1992, 8, 1043; Kanagasundaram et al., Biochim. Biophys. Acta, 1992, 1171, 198; Olsen et al., Mol. Endocrinol., 1991, 5, 1246; Saul et al., Appl. Environ. Microbiol., 1990, 56, 3117; Yaoita et al., Proc. Natl. Acad. Sci. USA, 1990, 87, 7090; Rogers et al., EMBO J., 1990, 9, 2273). In the context of the invention, “start codon” and “translation initiation codon” refer to the codon or codons that are used in vivo to initiate translation of an mRNA molecule transcribed from a gene encoding a JNK protein, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation region” refer to a portion of such an mRNA or gene that encompasses from about to-about 50 contiguous nucleotides in either direction (i.e., 5′ or 3′) from a translation initiation codon. Similarly, the terms “stop codon region” and “translation termination region” refer to a portion of such an mRNA or gene that encompasses from about 25 to about 50 contiguous nucleotides in either direction (i.e., 5′ or 3′) from a translation termination codon.

The present invention employs oligonucleotides for use in antisense modulation of DENN. Exemplary DENN antisense oligonucleotides that can be used in the methods of the invention have the following sequences:

| | |
|---|---|
| 5′-TCACTTGCCAGTCTCAAGCTG-3′: | SEQ ID NO: 1 |
| 5′-CCAGTCTCAAGCTGTTGGGCC-3′: | SEQ ID NO: 2 |
| 5′-GAACTTCTTCTTTTGCACCAT-3′: | SEQ ID NO: 3 |
| 5′-TCCAAGGGACAGGTACCTGTC-3′: | SEQ ID NO: 4 |
| 5′-GCTAGAGACAGGCCGGGGCGG-3′: | SEQ ID NO: 5 |

In the context of this invention, the term “oligonucleotide” refers to an oligomer or polymer of ribonucleic acid or deoxyribonucleic acid. This term includes oligonucleotides composed of naturally-occurring nucleobases, sugars and covalent intersugar (backbone) linkages as well as oligonucleotides having non-naturally-occurring portions which function similarly. Such modified or substituted oligonucleotides are often preferred over native forms because of desirable properties such as, for example, enhanced cellular uptake, enhanced binding to target and increased stability in the presence of nucleases.

An oligonucleotide is a polymer of a repeating unit generically known as a nucleotide. The oligonucleotides in accordance with this invention preferably comprise from about 8 to about 30 nucleotides. An unmodified (naturally occurring) nucleotide has three components: (1) a nitrogen-containing heterocyclic base linked by one of its nitrogen atoms to (2) a 5-pentofuranosyl sugar and (3) a phosphate esterified to one of the 5′ or 3′ carbon atoms of the sugar. When incorporated into an oligonucleotide chain, the phosphate of a first nucleotide is also esterified to an adjacent sugar of a second, adjacent nucleotide via a 3′-5′ phosphate linkage. The “backbone” of an unmodified oligonucleotide consists of (2) and (3), that is, sugars linked together by phosphodiester linkages between the 5′ carbon of the sugar of a first nucleotide and the 3′ carbon of a second, adjacent nucleotide. A “nucleoside” is the combination of (1) a nucleobase and (2) a sugar in the absence of (3) a phosphate moiety (Komberg, A., DNA Replication, W.H. Freeman & Co., San Francisco, 1980, pages 4-7). The backbone of an oligonucleotide positions a series of bases in a specific order; the written representation of this series of bases, which is conventionally written in 5′ to 3′ order, is known as a nucleotide sequence.

Oligonucleotides may comprise'nucleotide sequences sufficient in identity and number to effect specific hybridization with a particular nucleic acid. Such oligonucleotides which specifically hybridize to a portion of the sense strand of a gene are commonly described as “antisense.” In the context of the invention, “hybridization” means hydrogen bonding, which may be Watson-Crick, Hoogsteen or reversed Hoogsteen hydrogen bonding, between complementary nucleotides. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. An oligonucleotide is specifically hybridizable to its target sequence due to the formation of base pairs between specific partner nucleobases in the interior of a nucleic acid duplex. Among the naturally occurring nucleobases, guanine (G) binds to cytosine (C), and adenine (A) binds to thymine (T) or uracil (U). In addition to the equivalency of U (RNA) and T (DNA) as partners for A, other naturally occurring nucleobase equivalents are known, including 5-methylcytosine, 5-hydroxymethylcytosine (HMC), glycosyl HMC and gentiobiosyl HMC (C equivalents), and 5-hydroxymethyluracil (U equivalent). Furthermore, synthetic nucleobases which retain partner specificity are known in the art and include, for example, 7-deaza-Guanine, which retains partner specificity for C. Thus, an oligonucleotide's capacity to specifically hybridize with its target sequence will not be altered by any chemical modification to a nucleobase in the nucleotide sequence of the oligonucleotide which does not significantly effect its specificity for the partner nucleobase in the target oligonucleotide. It is understood in the art that an oligonucleotide need not be 100% complementary to its target DNA sequence to be specifically hybridizable. An oligonucleotide is specifically hybridizable when there is a sufficient degree of complementarity to avoid non-specific binding of the oligonucleotide to non-target sequences under conditions in which specific binding is desired, i.e., under physiological conditions in the case of in vivo assays or therapeutic treatment, and in the case of in vitro assays, under conditions in which the assays are performed.

Antisense oligonucleotides are commonly used as research reagents, diagnostic aids, and therapeutic agents. For example, antisense oligonucleotides, which are able to inhibit gene expression with exquisite specificity, are often used by those of ordinary skill to elucidate the function of particular genes, for example to distinguish between the functions of various members of a biological pathway. This specific inhibitory effect has, therefore, been harnessed by those skilled in the art for research uses. The specificity and sensitivity of oligonucleotides is also harnessed by those of skill in the art for therapeutic uses.

A. Modified Linkages: Specific examples of some preferred modified oligonucleotides envisioned for this invention include those containing phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioates and those with CH2 —NH—O—CH2, CH2 —N(CH3)—O—CH2 [known as a methylene(methylimino) or MMI backbone], CH2 —O—N(CH3)—CH2, CH2 —N(CH3)—N(CH3)—CH2 and O—N(CH3)—CH2 —CH2 backbones, wherein the native phosphodiester backbone is represented as O—P—O—CH2). Also preferred are oligonucleotides having morpholino backbone structures (Summerton and Weller, U.S. Pat. No. 5,034,506). Further preferred are oligonucleotides with NR—C(*)—CH2 —CH2, CH2 —NR—C(*)—CH2, CH2 —CH2 —NR—C(*), C(*)—NR—CH2 —CH2 and CH2 —C(*)—NR—CH2 backbones, wherein “*” represents O or S (known as amide backbones; DeMesmaeker et al., WO 92/20823, published Nov. 26, 1992). In other preferred embodiments, such as the peptide nucleic acid (PNA) backbone, the phosphodiester backbone of the oligonucleotide is replaced with a polyamide backbone, the nucleobases being bound directly or indirectly to the aza nitrogen atoms of the polyamide backbone (Nielsen et al., Science, 1991, 254, 1497; U.S. Pat. No. 5,539,082).

B. Modified Nucleobases: The oligonucleotides of the invention may additionally or alternatively include nucleobase modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-methylcytosine, 5-hydroxymethylcytosine (HMC), glycosyl HMC and gentiobiosyl HMC, as well synthetic nucleobases, e.g., 2-aminoadenine, 2-thiouracil, 2-thiothymine, 5-bromouracil, 5-hydroxymethyluracil, 8-azaguanine, 7-deazaguanine, N6 (6-aminohexyl)adenine and 2,6-diaminopurine (Kornberg, A., DNA Replication, W. H. Freeman & Co., San Francisco, 1980, pages 75-77; Gebeyehu, G., et al., Nucleic Acids Res., 1987, 15, 4513).

C. Sugar Modifications: Modified oligonucleotides may also contain one or more substituted sugar moieties. Preferred oligonucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C1 to C10 alkyl or C2 to C10 alkenyl and alkynyl. Particularly preferred are O[(CH2)n O]m CH3, O(CH2)n OCH3, O(CH2)n NH2, O(CH2)n CH3, O(CH2)n ONH2, and O(CH2)n ON[(CH2)n CH3)]2, where n and m are from 1 to about 10. Other preferred oligonucleotides comprise one of the following at the 2'position: C1 to C10 lower alkyl, substituted lower alkyl, alkaryl, aralkyl, O-alkaryl or O-aralkyl, SH, SCH3, OCN, Cl, Br, CN, CF, OCF3, SOCH3, SO2 CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes an alkoxyalkoxy group, 2′-methoxyethoxy (2′-O-CH2 CH2 OCH3, also known as 2′-O-(2-methoxyethyl) or 2′-MOE) (Martin et al., Helv. Chim. Acta, 1995, 78, 486-504). Further preferred modifications include 2′-dimethylaminooxyethoxy, i.e., a 2′-O(CH2)2 ON(CH3)2 group, also known as 2′-DMAOE and 2′-dimethylaminoethoxyethoxy, i.e., 2′—O—CH2 —O—CH2 —N(CH2)2.

Other preferred modifications include 2′-methoxy (2′-O-CH3), 2′-aminopropoxy (2′-OCH2 CH2 CH2 NH2) and 2′-fluoro (2'--F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,0531 5,639,873; 5,646,265; 5,658,873; 5,670,633; and 5,700,920, each of which is herein incorporated by reference.

D. Other Modifications: Similar modifications may also be made at other positions on the oligonucleotide, particularly the 3′ position of the sugar on the 3′ terminal nucleotide and the 5′ position of 5′ terminal nucleotide. The 5′ and 3′ termini of an oligonucleotide may also be modified to serve as points of chemical conjugation of, e.g., lipophilic moieties (see immediately subsequent paragraph), intercalating agents (Kuyavin et al., WO 96/32496, published Oct. 17, 1996; Nguyen et al., U.S. Pat. No. 4,835,263, issued May 30, 1989) or hydroxyalkyl groups (Helene et al., WO 96/34008, published Oct. 31, 1996).

Other positions within an oligonucleotide of the invention can be used to chemically link thereto one or more effector groups to form an oligonucleotide conjugate. An “effector group” is a chemical moiety that is capable of carrying out a particular chemical or biological function. Examples of such effector groups include, but are not limited to, an RNA cleaving group, a reporter group, an intercalator, a group for improving the pharmacokinetic properties of an oligonucleotide, or a group for improving the pharmacodynamic properties of an oligonucleotide and other substituents having similar properties. A variety of chemical linkers may be used to conjugate an effector group to an oligonucleotide of the invention. As an example, U.S. Pat. No. 5,578,718 to Cook et al. discloses methods of attaching an alkylthio linker, which may be further derivatized to include additional groups, to ribofuranosyl positions, nucleosidic base positions, or on internucleoside linkages. Additional methods of conjugating oligonucleotides to various effector groups are known in the art; see, e.g., Protocols for Oligonucleotide Conjugates (Methods in Molecular Biology, Volume 26) Agrawal, S., ed., Humana Press, Totowa, N.J., 1994.

Another preferred additional or alternative modification of the oligonucleotides of the invention involves chemically linking to the oligonucleotide one or more lipophilic moieties which enhance the cellular uptake of the oligonucleotide. Such lipophilic moieties may be linked to an oligonucleotide at several different positions on the oligonucleotide. Some preferred positions include the 3′ position of the sugar of the 3'terminal nucleotide, the 5′ position of the sugar of the 5′ terminal nucleotide, and the 2′ position of the sugar of any nucleotide. The N6 position of a purine nucleobase may also be utilized to link a lipophilic moiety to an oligonucleotide of the invention (Gebeyehu, G., et al., Nucleic Acids Res., 1987, 15, 4513). Such lipophilic moieties include but are not limited to a cholesteryl moiety (Letsinger et al., Proc. Natl. Acad. Sci. U.S.A., 1989, 86, 6553), cholic acid (Manoharan et al., Bioorg. Med. Chem. Let., 1994, 4, 1053), a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765), a thiocholesterol (Oberhauser et al., Nucl. Acids Res., 1992, 20, 533), an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651; Shea et al., Nucl. Acids Res., 1990, 18, 3777), a polyamine or a polyethylene glycol chain (Manoharan et al., Nucleosides & Nucleotides, 1995, 14, 969), or adamantane acetic acid (Manoharan et al., Tetrahedron Lett., 1995, 36, 3651), a palmityl moiety (Mishra et al., Biochim. Biophys. Acta, 1995, 1264, 229), or an octadecylamine or hexylamino-carbonyl-oxycholesterol moiety (Crooke et al., J. Pharmacol. Exp. Ther., 1996, 277, 923). Oligonucleotides comprising lipophilic moieties, and methods for preparing such oligonucleotides, are disclosed in U.S. Pat. Nos. 5,138,045, 5,218,105 and 5,459,255.

The present invention also includes oligonucleotides that are substantially chirally pure with regard to particular positions within the oligonucleotides. Examples of substantially chirally pure oligonucleotides include, but are not limited to, those having phosphorothioate linkages that are at least 75% Sp or Rp (Cook et al., U.S. Pat. No. 5,587,361) and those having substantially chirally pure (Sp or Rp) alkylphosphonate, phosphoamidate or phosphotriester linkages (Cook, U.S. Pat. Nos. 5,212,295 and 5,521,302).

E. Chimeric Oligonucleotides: The present invention also includes oligonucleotides which are chimeric oligonucleotides. “Chimeric” oligonucleotides or “chimeras,” in the context of this invention, are oligonucleotides which contain two or more chemically distinct regions, each made up of at least one nucleotide. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of antisense inhibition of gene expression. Cleavage of the RNA target can be routinely detected by gel electrophoresis and, if necessary, associated nucleic acid hybridization techniques known in the art. By way of example, such “chimeras” may be “gapmers,” i.e., oligonucleotides in which a central portion (the “gap”) of the oligonucleotide serves as a substrate for, e.g., RNase H, and the 5′ and 3′ portions (the “wings”) are modified in such a fashion so as to have greater affinity for the target RNA molecule but are unable to support nuclease activity (e.g., 2′-fluoro- or 2′-methoxyethoxy-substituted). Other chimeras include “wingmers,” that is, oligonucleotides in which the 5′ portion of the oligonucleotide serves as a substrate for, e.g., RNase H, whereas the 3′ portion is modified in such a fashion so as to have greater affinity for the target RNA molecule but is unable to support nuclease activity (e.g., 2′-fluoro- or 2′-methoxyethoxy-substituted), or vice-versa.

F. Synthesis: The oligonucleotides used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is also known to use similar techniques to prepare other oligonucleotides such as the phosphorothioates and alkylated derivatives.

### Bioequivalents

The compounds used in the methods of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to “prodrugs” and “pharmaceutically acceptable salts” of the oligonucleotides of the invention, pharmaceutically acceptable salts of such prodrugs, and other bioequivalents.

A. Oligonucleotide Prodrugs: The oligonucleotides of the invention may additionally or alternatively be prepared to be delivered in a “prodrug” form. The term “prodrug” indicates a therapeutic agent that is prepared in an inactive form that is converted to an active form (i.e., drug) within the body or cells thereof by the action of endogenous enzymes or other chemicals and/or conditions. For example, prodrug versions of the oligonucleotides of the invention can be prepared as SATE [(S-acetyl-2-thioethyl) phosphate] derivatives according to the methods disclosed in WO 93/24510 to Gosselin et al., published Dec. 9, 1993.

B. Pharmaceutically Acceptable Salts: The term “pharmaceutically acceptable salts” refers to physiologically and pharmaceutically acceptable salts of the oligonucleotides of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66, 1). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, malcic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

### ROUTES OF ADMINISTRATION

The methods of the invention contemplate using various routes for administering composition in accordance with the invention. Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Administration of the pharmaceutical compositions to practice the methods of the present invention can be carried out in a variety of conventional ways, such as oral ingestion, inhalation, topical application or cutaneous, subcutaneous, intraperitoneal, parenteral or intravenous injection. Intravenous administration to the patient is preferred.

Furthermore, one may administer the desired composition in a targeted drug delivery system, for example, in a liposome coated with a specific antibody, targeting, for example, neuronal tissue. The liposomes will be targeted to and taken up selectively by the afflicted tissue.

### GENE THERAPY

Oligonucleotides of the present invention can also be used for gene therapy for the treatment of neurodegenerative disorders characterized by abnormal expression, for example, overexpression, of DENN. Such therapy would achieve its therapeutic effect by introduction of the appropriate oligonucleotide (e.g., SEQ ID NO: 1) into cells of subjects having the oxidative stress disorder. Delivery of the oligonucleotide constructs can be achieved using a recombinant expression vector such as a chimeric virus or a colloidal dispersion system. An expression vector including the oligonucleotide sequence could be introduced to the subject's cells ex vivo after removing, for example, stem cells from a subject's bone marrow. The cells are then reintroduced into the subject, (e.g., into subject's bone marrow).

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), Rous Sarcoma Virus (RSV), and gibbon ape leukemia virus (GaLV), which provides a broader host range than many of the murine viruses. A number of additional retroviral vectors can incorporate multiple genes. All of these vectors can transfer or incorporate a gene for a selectable marker so that transduced cells can be identified and selected for. By inserting a hJIP-1/IB 1 sequence of interest into the viral vector, along with another gene which encodes the ligand for a receptor on a specific target cell, for example, the vector is now target specific. Preferred targeting is accomplished by using an antibody to target the retroviral vector. Those of skill in the art will know of, or can readily ascertain without undue experimentation, specific polynucleotide sequences which can be inserted into the retroviral genome to allow target specific retroviral vector containing the DENN antisense oligonucleotide.

Since recombinant retroviruses are defective, they require assistance to produce infectious vector particles. This assistance can be provided, for example, by using helper cell lines that contain plasmids encoding all of the structural genes of the retrovirus under the control of regulatory sequences within the LTR. These plasmids are missing a nucleotide sequence which enables the packaging mechanism to recognize an RNA transcript for encapsidation. Helper cell lines which have deletions of the packaging signal include but are not limited to .PSI.2, PA317 and PA12, for example. These cell lines produce empty virions, since no genome is packaged. If a retroviral vector in which the packaging signal is intact, but the structural genes are replaced by other genes of interest is introduced into such cells, the vector will be packaged and vector virions produced.

Another targeted delivery system for the DENN antisense oligonucleotide is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macromolecules. RNA, DNA and intact virions can be encapsulated within the aqueous interior and be delivered to cells in a biologically active form (Fraley, et al., Trends Biochem. Sci., 6:77, 1981). In addition to mammalian cells, liposomes have been used for delivery of polynucleotides in plant, yeast and bacterial cells. For a liposome to be an efficient gene transfer vehicle, the following characteristics should be present: (1) encapsulation of the genes of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6:682, 1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The surface of the targeted delivery system may be modified in a variety of ways. In the case of a liposomal targeted delivery system, lipid groups can be incorporated into the lipid bilayer of the liposome to maintain the targeting ligand in stable association with the liposomal bilayer. Various linking groups can be used for joining the lipid chains to the targeting ligand.

In general, the compounds bound to the surface of the targeted delivery system will be ligands and receptors which will allow the targeted delivery system to find and “home in” on the desired cells. A ligand may be any compound of interest which will bind to another compound, such as a receptor. In general, surface membrane proteins which bind to specific effector molecules are referred to as receptors. In the present invention, antibodies are preferred receptors. Antibodies can be used to target liposomes to specific cell-surface ligands. Preferred cell-surface ligands are those that are selectively expressed on neuronal tissues.

The present invention is illustrated in the following examples. Upon consideration of the present disclosure, one of skill in the art will appreciate that many other embodiments and variations may be made in the scope of the present invention. Accordingly, it is intended that the broader aspects of the present invention not be limited to the disclosure of the following examples.

### EXAMPLES

Materials and Methods:

Chemicals and Antibodies

DAPI (4,6-diamidino-2-phenylindole), Hoechst 33342, SB203580 (4-(4-fluorophenyl)-2-(4-methylsulfinylphenyl)-5-(4-pyridyl)-1H-imidazole) were obtained from Sigma Chemical Co. (St. Louis, Mo.). Propidium iodide (PI) was purchased from Bochringer Mannheim Co., (Indianapolis, Ind.). Staurosporine was obtained from Calbiochem (La Jolla, Calif.). 7-amino-actinomycin (7-AAD) Viaprobe, phycoerythrin (PE)-conjugated annexin V antibody and FADD monoclonal antibody were obtained from Pharmingen (San Diego, Calif.). Bradford dye reagent was purchased from Bio-Rad (Hercules, Calif.). GFP monoclonal antibody was purchased from Clontech (Palo Alto, Calif.). JNK3 and c-Jun (Ser73) polyclonal antibodies were obtained from Upstate Biotechnology Co. (Lake Placid, N.Y.). JNK1 (Fla.) polyclonal antibody (sc-571) and TRADD polyclonal (sc-7868) were obtained from Santa Cruz Biotechnology (Santa Cruz, Calif.). Monoclonal phospho-SAPK and MAPK p44/42 antibodies and polyclonal p38 MAPK antibody were purchased from Cell Signaling (Beverly, Mass.). Texas Red/FITC or Alexa488/562 conjugated secondary antibodies were obtained from Vector Labs (Burlingame, Calif.) or Molecular Probes (Eugene, OR), respectively. ECL (enhanced chemiluminescence) detection kit was obtained from Amersham Pharmacia Biotech (Piscataway, N.J.).

Plasmid Construction

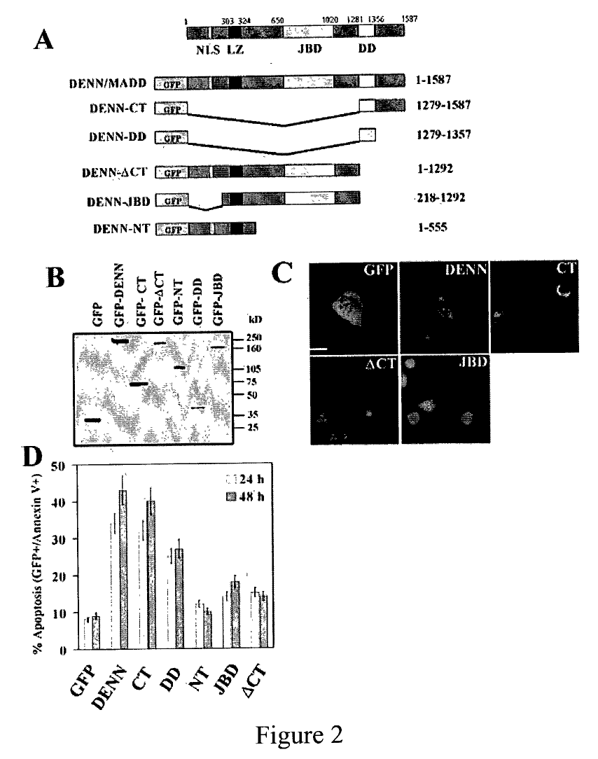

For constitutive overexpression under the cytomegaloviral promoter (CMV), green fluorescent protein (GFP)-DENN fusions were constructed by insertion of the full length coding region of DENN digested from pcDNA3-HA-DENN (Zhang et al., 1998) with HindIII/ApaI and insertion into the corresponding restriction sites of pEGFP-C3 (Clontech) to produce pGFP-DENN. Mutants of DENN were produced by pair-wise restriction digests of DENN with HindIII/Acc65I, HindIII/BamHI, or XhoI/Acc65I yielding the constructs: pGFP-ΔDD, pGFP-NT, pGFP-JBD, respectively. The DD containing region alone (residues 1279-1357) and the C-terminal DD-containing region (residues 1279-1587) were amplified by PCR with flanking EcoRI sites and inserted into pEGFP-C1. These produced the mutants pGFP-DD and pGFP-CT, respectively (FIG. 2A). All mutant constructs were sequenced by the Molecular Core Facility at the University of Southern California for proper in-frame insertions and absence of PCR introduced errors.

Cell Culture and Transfections NSC19 cells, a mouse-mouse neural hybrid cell line produced by fusing aminopterin-sensitive neuroblastoma (N18TG2) with mouse spinal cord (E1 2-14) motor neuron-enriched cells (Cashman et al., 1992), exhibit neuronal morphology with highly developed processes and express acetylcholine (Pedersen et al., 1999). In contrast to primary CNS cultures, NSC19 are more readily transfectable and highly responsive to apoptosis (Smirnova et al., 1998; Citron et al, 1997). Cells were maintained in high glucose Dulbecco's Modified Eagle's Medium (DMEM) (GIBCO-BRL/Life Technologies, Rockville, Md.) supplemented with 10% fetal bovine serum (FBS) and 1% streptomycin/penicillin. Neuro2A cells (CCL-131), C0S7 (CRL-1651) and human embryonic kidney 293 (CRL-1573) cells were acquired from ATCC. Murine neuroblastoma Neuro2A (N₂A) were grown in MEM supplemented with 10% FBS, while COS7 and 293 were maintained in DMEM supplemented with 10% FBS. Cells were plated (5 ×10⁵ cells/ml) in 35-mm dishes and transfected with 1 μg total DNA using Effectene or SuperFect (Qiagen) as described by the manufacturer. Efficiency of transfection, as visualized by epifluorescence microscope evaluation and FACS analysis, was approximately 10-30%. Stable transfectants were selected using 700 μg/ml G418 for 4-6 weeks. Approximately 80% of neuronal cells expressed GFP.

Primary mouse and rat hippocampal neurons were prepared from newborn C57B1/6 mouse or Sprague-Dawley rat pups. Briefly, hippocampi were dissected out, minced and digested with 100 U papain and 0.2 μg/ml DNaseI. Following digestion, cells were 'triturated with fire-polished Pasteur pipets and centrifuged in media containing 20 μg/ml BSA. Cells were plated on collagen-coated or poly-lysine coated plates at a density of 3×10⁶ cells/ml in Neurobasal medium with B27 supplement and antibiotics (Life Technologies, Gaithersburg, Md.). One day after plating, cells were exposed to 5 μM cytosine arabinoside for 4 d to kill dividing cells. Experiments were performed after 6-8 days in vitro (DIV).

β-Amyloid Treatment of Cells

Lyophilized, HPLC purified β-amyloid, Aβ₂₅₋₃₅, Aβ₁₋₄₀ , Aβ₁₋₄₂ and Aβ₄₀₋₁ were obtained from either Sigma Chemical Co., U.S. Peptide (Fullerton, Calif.) or Bachem (Torrance, Calif.), respectively. Peptides were reconstituted in double-distilled H₂O at 10 mM Aβ. To age the β-amyloid in vitro and produce aggregated, fibrillar Aβ, an aliquot of the Aβ stock solution was dissolved at the indicated concentration in culture medium and incubated at 37° C. for 3 to 5 days. Cells were incubated with the appropriate Aβ peptide concentration for the indicated times and examined for immunofluorescence or cell death assay. For experiments requiring co-treatment with SB203580, cells were treated for 2 days with 20 μM Aβ₍₁₋₄₀₎ and 10 μM SB203580 (solubilized in DMSO).

Cell Death Assays

Cells were analyzed by fluorescence-activated cell sorting (FACS) analysis on a FACSTAR PLUS (Becton-Dickinson) and analyzed with Cell Quest software. FACS analysis for two color analysis used GFP and either 7-amino-actinomycin (7-AAD) or propidium iodide

(PI) for cell death or GFP and phycoerythrin (PE)-conjugated annexin V for early apoptosis. The percentage of cell death was quantified as those cells expressing GFP and either annexin V or 7-AAD staining relative to total GFP expressing cells. Experiments were performed in triplicate with 10,000 events counted. TUNEL staining was performed according to manufacturer's instructions (Roche).

Western Immunoblotting and Kinase Assay

NSC 19 cells were harvested after the appropriate treatments by scraping into cold PBS and centrifuging at 1500 ×g for 5 min at 4° C. Cells were then lysed in buffer A (20 mM Tris-Cl, pH 7.5, 1% Triton X-100, 0.5% NP-40, 0.1 M NaCl, 20 mM β-glycerophosphate, 2 mM EDTA, 2 mM sodium pyrophosphate, 10% glycerol, 10 μg/ml leupeptin and 2 mM phenylmethylsulfonyl fluoride) for 20 min on ice followed by four 5-second sonication bursts on ice. Lysates were centrifuged at 14,000 ×g for 10 min at 4° C. Protein concentrations were measured by the Bradford (1976) assay using bovine serum albumin (BSA) standards. Approximately 50 μg of cell lysates were mixed with 2Xsample buffer and boiled for 5 min. Samples were resolved by 10% SDS-polyacrylamide gel electrophoresis (PAGE), transferred to nitrocellulose membranes, blocked in 5% skim milk and 1% BSA in 1XTBS, and incubated overnight at 4° C. with primary antibody at indicated dilutions. Blots were then washed with TBS buffer containing 0.15% Tween 20 (washing buffer), incubated with a 1:1000 dilution of anti-mouse or anti-rabbit IgG-conjugated horseradish peroxidase for 1 h at room temperature, washed again and processed by ECL detection (Amersham).

For kinase assays, cells were exposed to 25 μM Aβ₍₁₋₄₀₎ in the absence or presence of 1 μM CEP11004 for the specified duration. Cell extracts were prepared, incubated with 5 μl JNK antibody for 4 h at 4° C. and precipitated with 60 μl 50% slurry protein A/G sepharose overnight at 4° C. Immunoprecipitates were washed, then incubated with 1 μg GST-cJun (1-79) and 50 μM ATP in 40 μl for 30 min at 30° C. Reactions were terminated with 40 μl Laemmli buffer, boiled for 4 min, centrifuged and 40 μl was loaded onto 10% SDS-PAGE, transferred to nitrocellulose and probed with phospho-c-Jun (Ser73) antibody.

Immunohistochemistry and Immunocytochemistry

Human CNS tissues were obtained from the USC Alzheimer's Disease Research Consortium. Neuropathological examination confirmed AD using modified CERAD criteria (Mirra et al., 1991; The National Institute on Aging et al., 1997). Blocks of hippocampus (1 cm³) were dissected at autopsy and fixed in 10% phosphate-buffered formalin. Post-mortem intervals averaged 5 h. Paraffin-embedded sections (8 μm) of hippocampus, including entorhinal cortex, were stained with hematoxylin and eosin. Sections from four histologically normal, age-matched controls and four AD patients were immunostained using the Avidin-Biotinylated enzyme Complex (ABC) method (Vector Laboratories, Burlingame, Calif.). Sections were visualized with aminoethylcarbazole (AEC) (Zymed, So. San Francisco, Calif.) and a hematoxylin counterstain (Sigma). For fluorescence studies, hippocampal sections were heated to 95° C. in 6.0 M Citrate buffer for antigen retrieval, blocked in 10% Normal Goat Serum (NGS) and then immunostained using a cocktail of primary antibodies, followed by washes and incubation in secondary antibody cocktail (dilution 1:200) of anti-mouse conjugated to Alexa594 or anti-rabbit conjugated to Alexa488. Counterstain for nuclei was Hoechst 33342 at 5 μg/ml.

Fluorescence Microscopy

Cells were plated at a density of 5×10³ cells/ml in 8-well chamber slides (Fisher Co, Pittsburgh, Pa.) and transfected with 2 μg of the indicated plasmid DNA using Lipofectamine according to the manufacturer's protocol. Twenty-four or 48 h post-transfection, cells were washed in cold PBS, fixed in cold acetone/methanol (1:1) at −20° C. for 20 min, then mounted with Vectashield mounting medium containing DAPI or PI (Vector Laboratories). Images were captured on a Zeiss Axiophot microscope at 400X magnification using a SPOT digital camera integrated system. Photomicrographs were prepared using Adobe Photoshop 6.0. For confocal microscopy, cells were excited and imaged under Texas Red and FITC filters.

RT-PCR

Total RNA was purified using the RNeasy miniprep kit (Qiagen) and resuspended in 50 μt RNase free water. For each RT-PCR, 1 μg RNA was subjected to One-Step RT-PCR using Omniscript and Sensiscript Reverse transcriptases, HotStarTaq DNA polymerase and 0.5 μM of DENN primers: sense, SEQ ID NO: 6: 5′-CCGTGCCTCCCAGCATTGGCA-3′ and antisense, SEQ ID NO: 1: 5′-TCACTTGCCAGTCTCAAGCTG-3′, for 30 cycles with the following conditions: 94° C., 30 s; 60° C., 1 min; 72° C., 1 min. Human and murine actin prim were supplied by Clontech.

### RESULTS

### Example 1

DENN Overexpression Induces Neuronal Cell Death

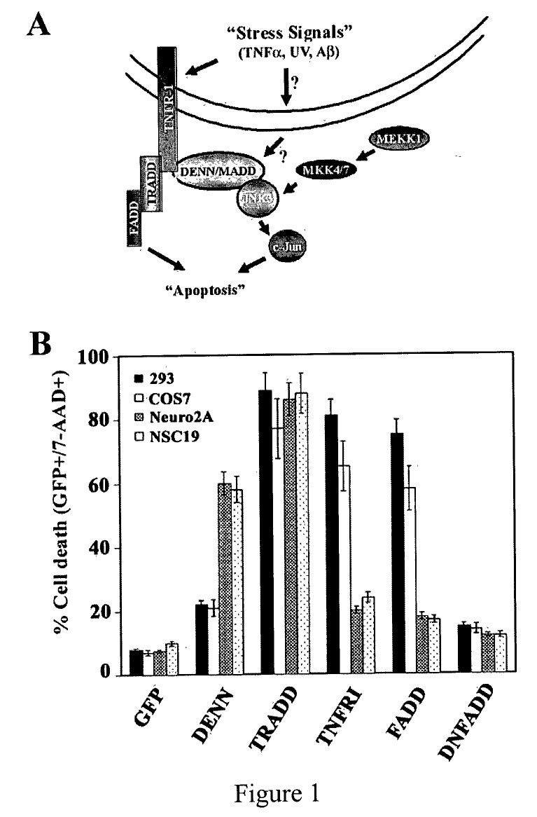

DENN was initially isolated by yeast two-hybrid analysis where it interacted with the neuronal specific JNK isoform, JNK3 (Zhang et al., 1998). DENN interacts with death domain (DD) containing proteins that link the TNF and JNK pathways and may be involved in the potentiation of cell death (FIG. 1A) (Schievella et al., 1997; Zhang et al., 1998; Wallach et al., 1999). To understand the functional significance of DENN expression in the JNK pathway, we overexpressed DENN and JNK3, as well as homologous DD containing proteins, in several cell lines. Neuroblastoma cultures (NSC19 and N₂A) and non-neuronal cells (human embryonic kidney 293 and COS7) were transiently transfected with either GFP or N-terminal GFP fusions of DENN, TRADD, FADD, TNFRI or dominant negative FADD (DNFADD) (FIG. 1B). A FLAG-tagged construct encoding the JNK3 gene was also included in the transfection, since 293 and COS7 cells do not express this protein isoform (data not shown). After 48 h, cells were collected and processed for flow cytometric analysis (FACS) using the nucleic acid dye 7-amino-actinomycin (7-AAD) to stain nonviable cells in the population of GFP expressing cells (Greeve et al., 2001).

DENN demonstrated significantly elevated cell death (approximately 60%) in neural cultures compared to non-neural cells (FIG. 1B). Previously, DENN was shown to promote the ERK and JNK pathways in COS cells with no observable cell death (Schievella et al., 1997). The 40 to 50% increase in cell death resulting from DENN overexpression in neuronal cultures compared to GFP transfectants suggests that DENN may participate in cell death pathways involving JNK. JNK3 overexpression alone did not promote cell death and DENN overexpression in the absence of overexpressed JNK3 showed only a 5-10% reduction in cell death potentiation (data not shown). These results suggest that N₂A and NSC 19 cells express endogenous levels of JNK3 that may be sufficient for transduction of DENN-induced cell death signals. Therefore, DENN overexpression more specifically induces cell death in neuroblastoma compared to non-neural cultures.

DENN overexpression induced cell death at levels comparable to homologous DD proteins, including TRADD and FADD. Induction of cell death in 293 and COS7 cells is mediated through overexpression of other DD proteins (Hsu et al., 1995; Hsu et al., 1996; Chang et al., 2001). Cells transfected with the DD containing adaptor proteins, TRADD, FADD or TNFRI yielded increased cell death in 293 and COS7 cells, with TRADD inducing approximately 80% and FADD resulting in 70% cell death. TRADD was especially cytotoxic in both neural and non-neural cultures (FIG. 1B). Although the intracellular domain of TNFRI induces apoptosis in rat PC12 cells (Haviv and Stein, 1998), our results revealed the full length form had little effect on mouse neuroblastoma cells, but induced 65 to 75% cell death of non-neural cultures. Expression of GFP and dominant negative FADD (DNFADD) served as controls for cytotoxicity resulting from GFP overexpression and FADD-mediated cell death, respectively, with minimal effects on cell viability.

### Example 2:

The C-terminal Domain of DENN is Required for Induction of Apoptosis

To determine the region of DENN required to promote cell death in neuronal cells, deletion mutants of DENN were constructed. The gene structure of DENN includes several functional domains: a putative nuclear localization signal (NLS), leucine zipper (LZ), JNK binding domain (JBD) and death domain (DD) (FIG. 2A). For convenience, we designate GFP fusion proteins, as shown in FIG. 2A, omitting the GFP portion of the chimera. Deletion mutants were constructed that encoded the C-terminal region containing the DD (DENN-CT), a smaller fragment solely encoding the death domain region (DENN-DD), a C-terminal truncation to delete the C-terminal region (DENN-ΔCT), the JNK binding domain region lacking the NLS and DD regions (DENN-JBD) and the N-terminal region containing the NLS and LZ (DENN-NT). These mutants were fused to the C-terminus of EGFP and individually overexpressed in munne neuronal NSCl9 cells.

Expression of GFP fusion proteins of DENN and the five deletion mutants was examined by Western blotting and fluorescence microscopy (FIG. 2B,C). Immunoblots using anti-GFP monoclonal antibodies confirmed protein expression at the appropriate molecular weights. Epifluorescence revealed the specific distribution of mutants in NSC19 cells. In surviving, transfected cells, DENN was localized throughout the cytoplasm and neurites. DENN-JBD and DENN-ΔCT were also localized to the cytoplasm and processes. Interestingly, surviving DENN-CT transfected cells resulted in pronounced cytotoxicity with cell rounding and perinuclear aggregates of the expressed protein in the reduced cytoplasm. This result suggests that although profound morphological changes occur prior to cell death and detachment, DENN-CT effects are within the cytoplasm, not requiring nuclear translocation.

Effects on cell survival resulting from overexpression of full length DENN were tested in both N₂A and NSC19 (FIG. 2D). To evaluate apoptosis in NSC19 cells, DENN and mutants of DENN were overexpressed and stained with phycoerythrin (PE)-conjugated annexin V and processed by two-color fluorescence activated cell sorting (FACS) (Schutte et al., 1998). Annexin V binds to externalized phosphatidylserine, and provides an early index of apoptosis (Rimon et al., 1997). Aβ treatment of human neuroblastoma cells produces phosphatidylserine externalization (Ekinci et al., 2000). As shown in FIG. 2D, at 24 and 48 h post-transfection, DENN exhibited a four-fold induction of apoptosis over GFP-transfected cells. Effects of DENN-CT were comparable to full length DENN and the shorter DD fragment also demonstrated a significant increase in apoptosis relative to GFP. The NT, JBD and ACT forms of DENN showed only modest increases in annexin V staining relative to vector control. These results suggest that the C-terminal region of DENN is critical for cell death signaling, although flanking regions may be required since the shorter DD fragment did not exhibit comparable annexin V staining as DENN-CT.

Example 3:

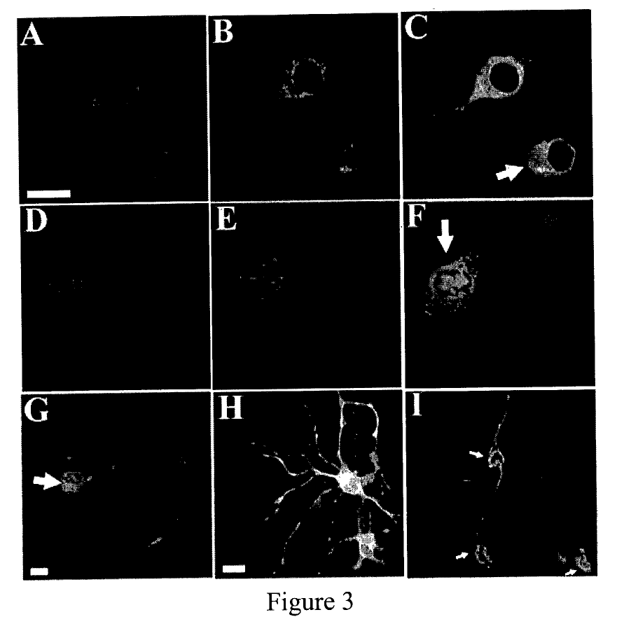

DENN co-localizes with activated JNK in vivo To investigate DENN and JNK localization, we examined immunocytochemically, the localization of DENN and JNK3 or activated JNK in neuroblastoma cells and primary neurons. In NSC19 cells, HA-tagged DENN and Flag-tagged JNK3 were co-expressed and examined by confocal microscopy. DENN, labeled with Texas Red (FIG. 3A), was diffusely distributed throughout the cytoplasm and neurites. Cell death was evident by the retraction of processes and cell rounding (FIG. 3C, arrow). JNK3 (FIG. 3B), labeled with FITC, shows diffuse cytoplasmic staining. With merged images, there was significant co-localization with DENN in cytoplasmic aggregates (FIG. 3C). Thus, overexpression of DENN and JNK3 reveals cytoplasmic distribution.

Furthermore, expression of endogenous DENN and activated JNK was examined in N₂A cells. In FIG. 3D-F, the localization of DENN, phospho-JNK and the merged image are shown, demonstrating co-localization in the nucleus (FIG. 3F, arrow). DENN has been shown to activate JNK and ERK in COS cells (Schievella et al., 1997), suggesting that co-localization and interaction between DENN and activated JNK in the nucleus may effect further downstream events. Although DENN does not promote cell death in COS cells, co-expression of JNK3 and DENN in COS cells demonstrated pronounced perinuclear concentration (FIG. 3G, arrow). Overexpression of DENN also localizes to the nucleus of these cells, similar to earlier reports from pyramidal neurons of patients with acute hypoxia (Zhang et al., 1998). These observations suggest that nuclear localization may not be causal to the cell death process.

Endogenous localization of DENN in primary cultures of mouse and rat hippocampal neurons was investigated (FIG. 3H,I). DENN localizes specifically to neurons as seen with double-labeling with DENN and MAP2 antibodies in FIG. 3H. In mouse hippocampal neurons, DENN was distributed in the cytoplasm and neuritic processes. Significant co-localization with activated JNK in the perinuclear region of neurons was observed (FIG. 3I, arrows). Interestingly, the JNK-binding, scaffold protein, JIP1, also accumulates in neurites of mouse hippocampal neurons under non-stressed conditions and exhibits punctate aggregates of perinuclear staining after ischemia induced by oxygen and glucose deprivation (Whitmarsh et al., 2001). Therefore, localization of DENN with JNK in the perinuclear region of hippocampal neurons is consistent with the co-localization observed with another JNK binding protein.

### Example 4:

DENN Overexpression Increases susceptibility of Neuronal Cells to Apoptotic Stimulation by Aβ Peptide

A significant role for JNK in neuronal cultures involves the transduction of stress signals evoked by Aβ. By exposing stable-transfected cell cultures to neurotoxic forms of Aβ, we examined whether DENN functions in neuronal apoptosis via JNK3. Previously, JNK and ERK have been shown to be activated by MADD overexpression in response to cytokine stimulation (Schievella et al., 1997; Al-Zoubi et al., 2001). The neurotoxic peptide fragment Aβ, a component of senile plaques, induces JNK activation leading to apoptosis in cortical and hippocampal neurons (Morishima et al., 2001). Moreover, the involvement of FasL/FAS receptor and p75 NTR in JNK-induced apoptosis implicates death domain-containing factors mediated through cell death receptors (Harrington et al., 2002; Morishima et al., 2001).

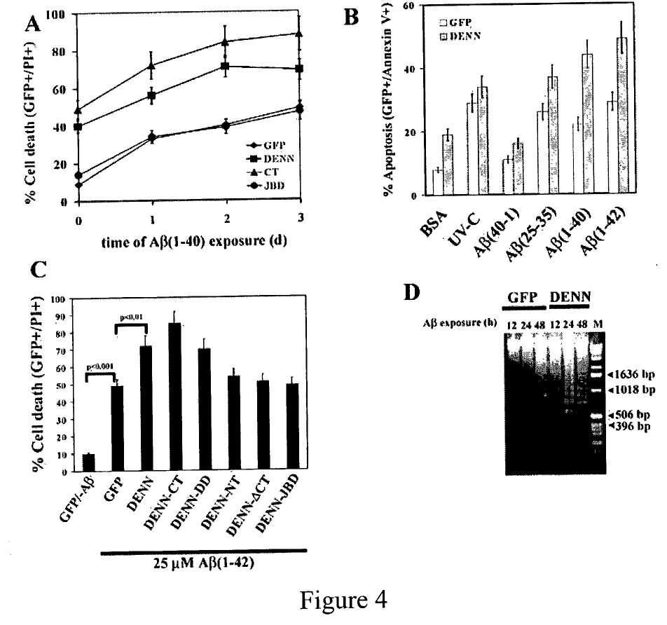

Aβ is selectively neurotoxic to a variety of neuronal subpopulations, including cells of the neocortex and hippocampus, as well as neuronal cell lines (Pike et al., 1991; Li et al., 1996; Ii et al, 1996; Estus et al., 1997; Kruman et al., 1997; Troy et al., 2000; Ekinci et al., 2000). The precise toxic peptide form of Aβ is controversial, with in vitro studies using Aβ₂₅₋₃₅, Aβ₁₋₄₀ and Aβ₁₋₄₂ (Harada and Sugimoto, 1999; Troy et al., 2000; Ekinci et al., 2000). The concentration and fibrillar state of Aβ are important considerations. NSC 19 and N₂A cells were sensitive to 25 μM Aβ₁₋₄₀, pre-incubated at 37° C. to form fibrils. In FIG. 4A, NSC19 cells expressing GFP fused mutants of DENN were exposed to 25 μM Aβ₍₁₋₄₀₎ for various time intervals and assessed for cell death by FACS analysis of PI uptake. Transfected cells gated for GFP expression showed increased cell death with prolonged Aβ exposure in all cultures. After 2 and 3 days of Aβ exposure, approximately 70-80% of cells overexpressed DENN and DENN-CT, respectively, while about 35-40% of DENN-JBD and GFP-transfected cells were dead.

Exposure to Aβ₁₋₄₀ dramatically increased apoptosis in DENN-transfected cells compared to untreated, control transfectants (FIG. 4B). Apoptosis induced by DENN overexpression was assessed by annexin V staining of cultures after exposure to a potent, JNK-activating stimulus (UV irradiation) or to various forms of Aβ peptide, including Aβ(25-35), Aβ(1-40) and Aβ(1-42) or the reverse peptide Aβ(40-1). In all cases, peptides were incubated at 25 μM for 3 days to promote the fibrillar state and then added to NSC19 cultures expressing either GFP or GFP-DENN, and then harvested after 48 h. UV irradiation resulted in similar levels of apoptosis in both the GFP and DENN transfected cultures (FIG. 4B), suggesting UV stimulation functions by activating additional apoptotic pathways acting independently of DENN or JNK-mediated pathways. Response to the reverse Aβ peptide was similar to the control BSA, with DENN overexpression alone demonstrating a 2-fold increase in annexin staining. However, Aβ exposure enhanced apoptosis exclusively in DENN-transfected cells above that of control cultures (p<0.005). Cultures exposed to Aβ₍₁₋₄₂₎ and Aβ(1-40) specifically promoted increased apoptosis in DENN-transfected cultures. Therefore, Aβ acts synergistically with DENN expression to induce apoptosis exceeding that of either Aβ treatment or DENN overexpression alone.

Neurotoxicity induced by Aβ₍₁₋₄₀₎ exposure was further examined using clonal cells expressing GFP-fused mutant forms of DENN. Cells were transiently transfected and exposed to Aβ for 48 h. There was a five-fold increase in cell death observed after Aβ exposure with GFP-expressing cultures, with a further increase of 20% in cell death with DENN overexpression (FIG. 4C). The C-terminal DENN mutants, DENN-CT and DENN-DD, also induced considerable cell death with approximately 30% and 20% increases, respectively, above vector control cells. DENN-NT, DENN-ΔCT and DENN-JBD did not augment cell death above vector controls, suggesting that these DENN mutants lack a critical region for induction of cell death.

Primary cultures of rat cortical neurons demonstrate DNA fragmentation, an indicator of apoptosis, after Aβ₍₂₅₋₃₅₎ exposure (Harada and Sugimoto; 1999). N₂A cells also responded to Aβ toxicity enhanced by DENN overexpression. Responses of GFP and DENN transfected clones of N₂A cells exposed to Aβ₍₁₋₄₀₎ were monitored over time. Cells were exposed to Aβ₍₁₋₄₀₎ for up to 48 h and their DNA extracted and analyzed by agarose gel electrophoresis (FIG. 4D). DNA fragmentation did not occur in GFP-expressing cells until 48 h exposure to Aβ, when cells revealed neurite retraction.

DNA fragmentation is a late event in cell death, and occurs in GFP-expressing cells concomitant with significant cell damage. However, in DENN-expressing cells, DNA fragmentation was detected as early as 12 hours after Aβ addition, and laddering increased thereafter. These findings are consistent with the accelerated induction of cell death seen with DENN overexpression. Taken together, DENN overexpression synergistically enhances Aβ toxicity in neuronal cells.

### Example 5:

DENN Expression Activates the JNK Pathway in Neuronal Cultures

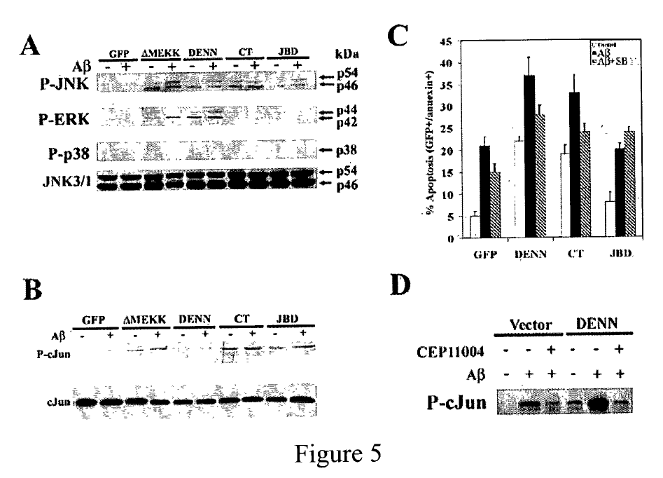

DENN specifically interacts with JNK and synergistically with Aβ to promote cell death. JNK and c-Jun phosphorylation are up-regulated in PC12 cells and primary cortical cultures in response to Aβ (Troy et al., 2001; Morishima et al., 2001). To determine whether MAPK/SAPK activation occurs with DENN overexpression and Aβ exposure in neuronal cultures, we performed Western immunoblotting using phospho-specific antibodies to JNK, ERK and p38 (FIG. 5A). After 24 h of Aβ exposure, NSC19 clones expressing GFP, ΔMEKK1, DENN or DENN mutants, DENN-CT and DENN-JBD, were probed. The 46 kD and 54 kD bands, corresponding to p46 and p54 isoforms of phosphorylated JNK, were increased in ΔMEKK1, DENN, DENN-CT and DENN-JBD cultures relative to control, confirming JNK activation. DENN and DENN-JBD affected 3 to 4-fold increase in the p54 band with Aβ exposure, suggesting interaction with DENN further enhanced Aβ-induced activation of JNK. Similar increases were observed with the constitutively active MEKK1 (ΔMEKK1), an upstream activator of JNK, while GFP-expressing cultures showed little increase of phospho-JNK at 24 h. In contrast, the overall levels of total JNK3 and JNK1 expression remained unchanged.

Further examination of activation of phosphorylated MAPKs revealed that DENN induced activation of ERK1 (p44) and ERK2 (p42) (FIG. 5A). Aβ exposure increased the level of phospho-ERK, while induction of ERK by ΔMEKK1 was minimally enhanced by Aβ. Additionally, p38 phosphorylation was not observed in cultures overexpressing DENN or by treatment with Aβ.

We next examined whether DENN overexpression along with Aβ exposure affected c-Jun phosphorylation. Expression of ΔMEKK1, DENN, DENN-CT and DENN-JBD showed sustained levels of phosphorylated c-Jun, as detected on immunoblots with anti-phosphorylated c-Jun (Ser73) (FIG. 5B). With Aβ₍₁₋₄₂₎ exposure, c-Jun phosphorylation by DENN was increased relative to untreated, but was less than the expression levels seen with ΔMEKK1, DENN-CT and DENN-JBD expression. The C-terminal of DENN has previously been shown to activate the JNK pathway (Schievella et al., 1997) and DENN-JBD may therefore promote c-Jun phosphorylation by binding to JNK and potentiating activity. GFP transfected cell cultures demonstrated minimal c-Jun phosphorylation, although with Aβ exposure, phosphorylated c-Jun was elevated, suggesting that while JNK activation is minor, c-Jun phosphoryation is elevated. Western analysis of total c-Jun protein remained constant under all conditions (FIG. 5B).

### Example 6:

Aβ-induced Apoptosis is Attenuated by JNK Inhibitors in DENN Overexpressing Cells

To determine whether the cell death response is affected by inhibiting the JNK pathway, NSC19 cells overexpressing DENN or mutants of DENN were treated with the pyridinyl imidazole p38/JNK inhibitor SB203580 (Eilers et al., 2001) in the presence or absence of pβ₍₁₋₄₀₎. At a concentration of 1 μM, SB203580 inhibits p38 SAPK. However, at 10 μM, JNK activity is also inhibited (Han et al., 1999; Brinkman et al., 1999). Cells stained with annexin V and analyzed by flow cytometry revealed that cells exposed to both the inhibitor and Aβ demonstrated a significantly attenuated apoptotic response maximal in cells overexpressing either DENN or DENN-CT (p<0.01) (FIG. 5C). Interestingly, DENN-JBD transfected cells revealed a reproducible increase in cell death with Aβ/SB203580 co-treatment, (p<0.05). DENN-JBD binding to JNK3 may ablate SB203580 inhibition, suggesting the DENN binding site of JNK may partially overlap that of the SB203580 compound and that full length DENN contains additional regions that either promote or fail to interfere with SB203580 inhibition.

As further evidence of DENN involvement in JNK activation, we examined whether JNK activity was affected by the indolocarbazole JNK inhibitor CEP11004, a derivative of CEP1347, shown to promote neuronal survival and inhibit JNK3 activation (Maroney et al., 1998; Namgung and Xia, 2000; Bozyczko-Coyne et al., 2001). N₂A clones expressing GFP or DENN were exposed to 25 μM Aβ₍₁₋₄₂₎ either in the absence or presence of 1 μM CEP11004. Previously, inhibitiori of Aβ-induced cell death in neuronal PC12 cells and sympathetic neurons required less than 300 nM CEP1347 (Troy et al., 2001). After 24 h Aβ exposure, JNK was immunoprecipitated from cell extracts and incubated with GST-c-Jun substrate and ATP in an in vitro kinase assay. The reactions were immunoblotted with antibody specific to phosphorylated c-Jun. As shown in FIG. 5D, JNK activity was increased by Aβ exposure in both GFP and DENN transfected cell cultures. However, DENN overexpressing cells exhibited elevated constitutive JNK activity in the absence of Aβ and a two-fold higher induction of JNK activity upon Aβ exposure. Addition of CEP11004 significantly reduced JNK activity by four-fold in both cell cultures indicating that CEP11004 attenuated both Aβ-mediated and DENN-induced JNK activity and c-Jun phosphorylation. Thus, reduction of cell death and JNK activity by JNK inhibitors in DENN overexpressing cell cultures supports DENN involvement in c-Jun activation and cellular apoptosis.

### Example 7:

Aβ Decreases Endogenous DENN Expression in Neuronal Cultures

Since overexpression of DENN activates cell death through JNK mediation, the impact of endogenous DENN on cell survival was investigated. DENN homologs in rodent and C. elegans have previously suggested a role in Ca²⁺ -dependent exocytosis and synaptic vesicle release through Rab3 association (Oishi et al., 1998; Iwasaki et al., 1997). Amyloid Precursor Protein (APP) is internalized and released through vesicular trafficking (Marquez-Sterling et al., 1996) and DD containing factors, including p75 neurotrophin receptor (NTR) and Par4 are implicated in neuronal death in AD (Perini et al., 2002; Guo et al., 1998).

Protein and RNA expression of neuroblastoma cultures treated with Aβ and immunostaining of rat hippocampal neurons revealed decreased endogenous DENN expression.

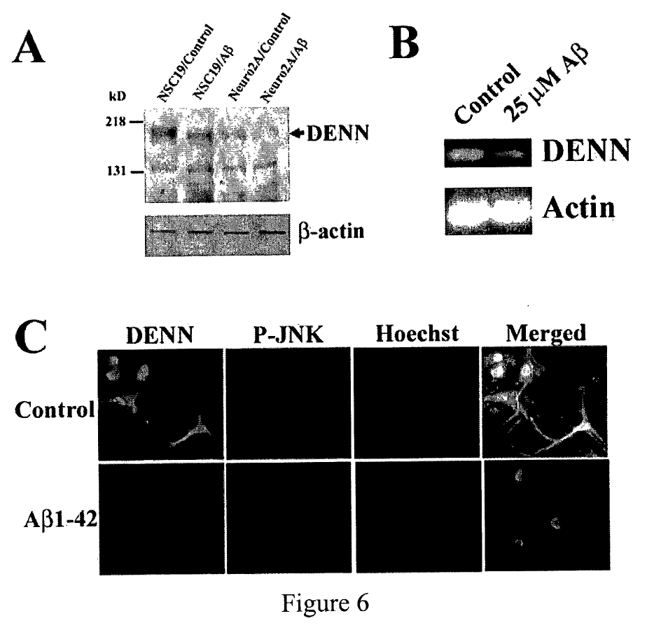

In FIG. 6A, endogenous DENN was significantly decreased by Aβ exposure in both NSC19 and N₂A cells. In untreated cultures, NSC19 cells expressed more DENN relative to N₂A. However, Aβ reduced DENN in both neuronal cell types by approximately 3-fold, while actin protein remained relatively constant. Cultures treated with additional factors which induce oxidative stress, including hydrogen peroxide and glutamate, also demonstrated reduction in DENN expression (data not shown).

RNA expression was compared in N₂A cells (FIG. 6B). RT-PCR of Aβ exposed cultures using DENN gene specific primers indicated a four-fold reduction in DENN expression. RT-PCR of β-actin remained constant indicating that the reduction in DENN RNA was not a result of cell loss. Previously, in AIDS-KS cells, there was complete and selective inhibition of DENN expression by actinomycin D treatment (Murakami-Mori et al., 1999). Reduction in DENN expression therefore directly correlates with cell survival suggesting that DENN is either affected by apoptotic factors or functions to promote cell survival.

Endogenous DENN expression was examined in rat hippocampal neurons exposed to Aβ₍₁₋₄₂₎ in relation to activation of JNK. In FIG. 6C, DENN expression is reduced in Aβ treated cultures relative to control. Concomitant with reduced DENN expression is increased nuclear localization of phospho-JNK in Aβ exposed cultures. DENN is excluded from the nucleus in these cultures, suggesting that JNK nuclear translocation is potentiated by a reduction in the cytoplasmic pool of DENN. In control cultures, DENN and phosphor-JNK co-localize in the cytoplasm, while the remaining reduced DENN expression in Aβ exposed cultures remains predominantly perinuclear and does not co-localize with nuclear phosphor-JNK.

### Example 8:

DENN Expression is Reduced in Human Hippocampus with AD Pathology

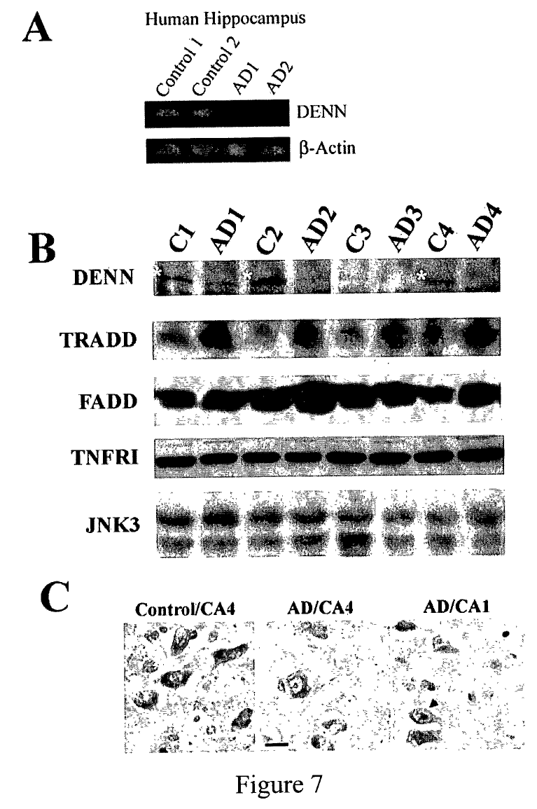

To examine endogenous DENN expression in an environment of long-term beta amyloid accumulation, tissue from AD patients was compared to controls for RNA levels. RT-PCR of human hippocampal tissues from two age-matched controls and two patients diagnosed with AD demonstrated a dramatic reduction in DENN expression, while β-actin remained at similar levels in both control and AD (FIG. 7A). This result suggests a correlation between reduced DENN expression and AD pathogenesis.

To determine the distribution of DENN protein in human hippocampus, tissue homogenates were examined by Western blotting. Polyclonal anti-DENN antibody, generated against synthetic peptides including the C-terminus, detected a major band at 140 kDa (Zhang et al., 1998; Chow and Lee, 1996; Chow et al., 1998). We compared protein expression in four age-matched controls and four AD hippocampi using antibodies specific to DENN, TRADD, FADD, TNFRI and JNK3. In both control and AD cases, JNK protein levels were similar (FIG. 7B). Death domain proteins in the CNS have homology to DENN (Schievella et al., 1997; Wallach et al., 1999). FADD and TNFRI levels were relatively similar in both AD and controls. However, TRADD protein was increased in AD hippocampal homogenates. One possibility is that with the influx of macrophages to remove damaged neurons, .TRADD is activated and recruited to the TNF receptor in response to TNF activation. Significantly, the 140 kDa band of DENN showed less expression in AD homogenates than controls although JNK3 remained unchanged suggesting that DENN or a fragment of DENN may be involved in essential functions of hippocampal neurons which have been impaired by oxidant stress and neuronal dysfunction characteristic of AD pathogenesis.

Reduced DENN expression was further confirmed by immunohistochemical analysis of control and AD tissue in CA4 and CA1 regions of the human hippocampus. As seen in FIG. 7C, neurons in AD-affected regions of the hippocampus showed significantly less cytoplasmic staining in areas CA4 and CA1. Some rare neurons also showed nuclear staining which was previously seen in pyramidal neurons of patients with acute hypoxia (Zhang et al., 1998).

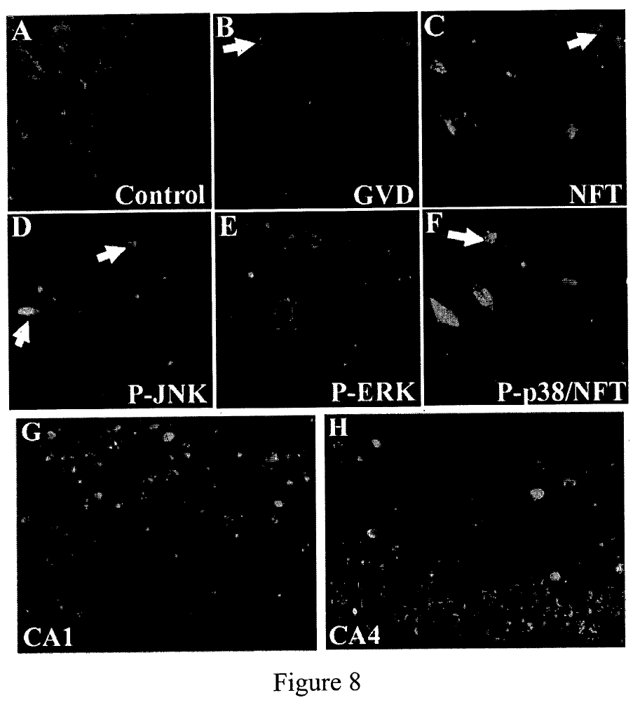

To determine effects of AD related neuronal pathology to DENN expression, CA1 neurons of the hippocampus were immunostained with markers of AD pathogenesis, including hyperphosphorylated tau (MAb AT8) and granulovacuolar degeneration, MAb 3A4. Normal control expression of DENN in CA1 of human hippocampus revealed cytoplasmic localization in pyramidal neurons. Double inmmunofluorescence with anti-DENN (green) showed reduced DENN expression in regions of granulovacuolar degeneration (red, punctate) (FIG. 8B) and neurofibrillary tangles (NFTs) (FIG. 8C). Similar to rat hippocampal neurons, DENN expression in AD is dramatically reduced in areas with phospho-JNK immunoreactivity (FIG. 8D,G,H). DENN staining (green) is significantly decreased in CA1 (FIG. 8G), while phospho-JNK (red) is distributed widely. Staining of DENN in the CA4 region and dentate gyrus is stronger (FIG. 8H), with less phospho-JNK staining. Minor staining was observed with DENN and phospho-ERK (FIG. 8E), suggesting reduced DENN expression is may be essential for JNK activation rather than ERK activation. In addition, as a control for MAPK activation and AD pathology, we observed distinct nuclear localization of activated p38 SAPK (p38-P) in AD hippocampal neurons also containing phosphorylated tau (FIG. 8F) (Atzori et al., 2001;Ferrer et al., 2001).

### Example 9:

Inhibition of Endogenous DENN Induces Cell Death and Enhances Aβ Cytotoxicity

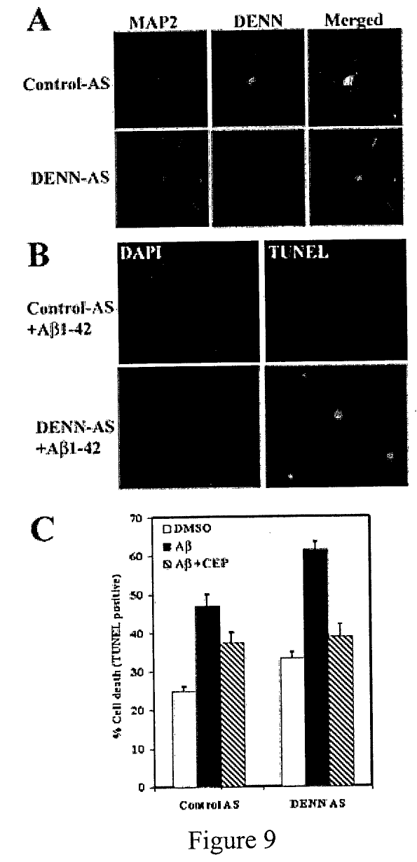

Because DENN protein expression is dramatically reduced in response to oxidative stress factors in both cultured neurons and human brain, we examined the effect of inhibiting DENN expression in mouse primary hippocampal neurons. Using anti-sense oligonucleotides designed against unique sequences of the DENN JBD region, we assessed the effects of DENN inhibition and Ab exposure in the absence of DENN expression. In FIG. 9A, double staining with the neuronal marker MAP2 and DENN revealed inhibition of DENN expression with the DENN anti-sense (DENN-AS), while treatment with Control-AS, an oligonucleotide with a scrambled sequence of the DENN sequence, still expressed DENN. However, surviving neurons treated with DENN-AS maintain a low level of DENN expression.

To assess cell death in neuronal cultures exposed to anti-sense oligonucleotides and Aβ, TUNEL staining was performed (FIG. 9B). Exposure to 25 μM Aβ resulted in increased TUNEL staining in both Control and DENN AS cultures. However, due to the increased TUNEL staining with DENN-AS treated cultures, the overall cell death with Aβ was 10-15% greater in these cultures (FIG. 9C). DENN-AS treatment alone increased cell death by 20%. This suggests that the toxicity produced by DENN inhibition and Aβ toxicity is additive. Furthermore, the level of cell death observed with Aβ was abrogated by exposing neuronal cultures to 1 βM CEP11004. This suggests that diminished DENN expression mediates cell death differently from overexpression, since the JNK inhibitor has the effect of reducing Aβ-induced JNK activation and cell death, but has little effect on DENN AS-induced cell death.

### DISCUSSION

The above examples demonstrate that DENN function differs under overexpression versus reduced expression conditions. Overexpression induces apoptosis selectively in neuronal N₂A and NSC19 cells, but not in 293 or COS7 non-neuronal cultures. As a member of the family of proteins containing death domains, DENN was most similar to TRADD, an adaptor protein in the TNFR linked pathway, in potentiating an apoptotic response in neuroblastoma cultures. The C-terminal region of DENN includes a death domain region with 15% homology to TRADD, and is indispensable for cell death. Thus, the invention provides methods for treating neurodegenerative diseases using antagonists that bind to the C-terminal region of DENN.

The examples also demonstrate that JNK activity and cell death enhanced by DENN overexpression is diminished in the presence of JNK inhibitors, SB203580 and CEP1004. This establishes a link in the death paradigm connecting DENN expression and JNK-c-Jun activation.

In cell culture, Aβ exposure of NSC19 and N₂A cells decreased endogenous DENN protein and RNA expression, further suggesting a role for DENN in Aβ-mediated neurotoxicity. However, neuronal cultures overexpressing DENN showed enhanced susceptibility to Aβ neurotoxicity with increased JNK activity and cell death. JNK inhibitors were insufficient to completely abrogate Aβ-induced cell death. DENN-induced neurotoxicity functions primarily through the JNK pathway, but with little involvement of p38. Moreover, ERK activation was observed in DENN overexpressing cells suggesting additional MAPK pathways are activated by DENN and may function independently to promote survival under stress conditions.

In the AD-affected human hippocampus, reduced DENN protein and RNA expression suggests DENN function may be critical for regulation of neuronal survival. Western blot analysis and RT-PCR confirmed that DENN expression was consistently higher in CNS tissues from normal controls relative to AD. While seemingly contradictory to DENN overexpression, a threshold of constitutive DENN expression may be essential for neuronal survival. Thus, the examples establish that modulation of DENN expression is a route to treatment of neuronal disorders.

Members of the JNK and TNF pathways, JNK3 and TNFRI, respectively, which interact with DENN, each revealed stable protein expression in controls compared to AD tissues. In contrast, although protein expression of DENN was decreased in the AD hippocampus, related death domain containing proteins, such as TRADD and FADD were increased or remained unchanged, respectively.

Immunocytochemical analysis of regions of the hippocampus, including vulnerable and affected CA1 pyramidal neurons, support evidence of a decrease in DENN expression in AD and indicate some nuclear translocation of DENN. While immunoblots of hippocampal homogenates indicated JNK expression remained constant in control versus AD, immunocytochemistry revealed high levels of constitutively activated MAPKs in controls. In these cases, DENN co-localized with phospho-JNK and phospho-ERK within neuronal soma. In AD, co-localization of activated JNK with DENN was primarily intranuclear, while activated ERK did not co-localize with DENN.

Under normal conditions, DENN is abundantly expressed in the cytoplasm of hippocampal neurons of CA1, CA3 and CA4. In AD, reduced DENN expression may be neuroprotective or conversely occur as a consequence of AD pathology. Phosphorylated tau (MAb AT8) and granulovacuolar degeneration (GVD) (MAb 3A4), are widely expressed throughout CA1 in AD hippocampus. Neurons with AT8 immunostaining show less DENN expression, while neighboring neurons lacking neurofibrillary tangles (NFTs) were intensely stained. Furthermore, there is virtually a complete absence of co-staining of DENN with the GVD marker previously linked to early AD neuronal changes. Such correlations between AD neuropathology and DENN expression suggest a neuroprotective role for DENN.

However, overexpression of either normal or deletion mutants of DENN lead to JNK and ERK activation in neuroblastoma cultures derived from sympathetic and motor neuron hybrid lineages. Aβ exposure combined with DENN overexpression further enhances phosphorylation of specific JNK and ERKs. These cells are the most susceptible to apoptosis and in vitro demonstrate significant cell death resulting in annexin V staining, DNA laddering and PI uptake. The most compelling evidence indicting JNK activation leading to neuronal apoptosis is suggested by JNK3−/− mice which are resistant to kainic acid induced excitotoxicity, Aβ exposure and NGF-induced sympathetic neuron cell death.

Oxidative stress resulting from beta amyloid deposits in senile plaques as well as effects of abnormal tau phosphorylation further challenge CA1 vulnerable neurons and activating mitogenic and cellular stress signaling. The examples show phosphorylated p38 co-localized to neurons bearing NFTs, a finding observed by others (Ferrer et al.,2001; Atzori et al., 2001). In these and neighboring neurons, p38 is localized within nuclei. MAPKs, p38 and JNK, phosphorylate transcription factors, such as ATF2, c-Jun and c-Fos and death-inducing genes, including TNF-α, FasL and DP5, are activated by these transcription factors (Estus et al., 1997; Morishima et al., 2001; Imaizumi et al., 1999). Surprisingly, overexpression of DENN resulted in a CRE-dependent induction of TNF-α.

The constitutive levels of specific signal transduction molecules in the hippocampus may set the thresholds for induction of neuronal apoptosis or cell survival. JNK3 and the FAS related death domain adapter protein, FADD/MACH/FLICE, exhibited similar protein expression in both control and AD homogenates. JNK levels also remain constant in AD and age-matched controls and FAS/FasL are not believed to affect AD vulnerable neurons. However, DENN protein expression was reduced in AD. Thus, DENN under “healthy” cell conditions functions in essential cellular function, but under stressed conditions initiated by cell surface death signals, DENN may be a pivotal link to intracellular signaling cascades involved in apoptosis. Conversely, the induction of cell death by specific signals from the surface may promote reduction of several constitutive proteins, including DENN, critical for cellular function. The examples have demonstrated that DENN is a molecule that when overexpressed, exhibits toxicity to the cell, but when inhibited is also harmful.

All of the publications which are cited in the body of the instant specification or listed in the attached list of references are hereby incorporated by reference in their entirety.

It is also to be appreciated that the foregoing description of the invention has been presented for purposes of illustration and explanation and is not intended to limit the invention to the precise manner of practice herein. It is to be appreciated therefore, that changes may be made by those skilled in the art without departing from the spirit of the invention and that the scope of the invention should be interpreted with respect to the following claims.

References:

Many of the references are cited in full in the main body of the specification. The following list is provided for references not cited in full in the main body of the specification.

1. Abe K, Saito H (2000) Amyloid beta neurotoxicity not mediated by the mitogen-activated protein kinase cascade in cultured rat hippocampal and cortical neurons. Neurosci Lett Sep 29;292(1):1-4

2. Adams, J H, Duchen, L W (1992) Greenfield's neuropathology, 5^{th} Edn. Oxford University Press, New York, pp 1312-1314.

3. Al-Zoubi A M, Efimova E V, Kaithamana S, Martinez O, El-Idrissi Mel-A, Dogan R E, Prabhakar B S (2001) Contrasting effects of IG20 and its splice isoforms, MADD and DENN-SV, on tumor necrosis factor alpha-induced apoptosis and activation of caspase-8 and -3. J Biol Chem 276(50):47202-11.

4. Anderson A J, Su J H, Cotman C W (1996) DNA damage and apoptosis in Alzheimer's disease: colocalization with c-Jun immunoreactivity, relationship to brain area, and effect of postmortem delay. J Neurosci 16(5):1710-9.

5. Atzori C, Ghetti B, Piva R, Srinivasan A N, Zolo P, Delisle M B, Mirra S S, Migheli A (2001) Activation of the JNK/p38 pathway occurs in diseases characterized by tau protein pathology and is related to tau phosphorylation but not to apoptosis. J Neuropathol Exp Neurol 60(12):1190-7.

6. Baker S J, Reddy E P (1998) Modulation of life and death by the TNF receptor superfamily. Oncogene 17(25):3261-70.

7. Behrens A, Sibilia M, Wagner E F (1999) Amino-terminal phosphorylation of c-Jun regulates stress-induced apoptosis and cellular proliferation. Nat Genet 21(3):326-9.

8. Bozyczko-Coyne, D, O'Kane, T M, Wu, Z-L, Dobrzanski, P, Murthy, S, Vaught, J L, Scott, R W (2001) CEP-1347/KT-7515, an inhibitor of SAPK/JNK pathway activation, promotes survival and blocks multiple events associated with Aβ-induced cortical neuron apoptosis. J Neurochem 77(3):849-63.

9. Bradford M M (1976) A rapid and sensitive method for the quantitation of microgram quantities of protein utilizing the principle of protein-dye binding. Anal Biochem 72:248-54.

10. Brinkman B M N, Telliez J-B, Schievella A R, Lin L-L, Goldfeld A E (1999) Engagement of tumor necrosis factor (TNF) receptor 1 leads to ATF-2- and p38 mitogen-activated protein kinase-dependent TNF-α gene expression. J Biol Chem 274(43):30882-30886.

11. Cashman N R, Durham H D, Blusztajn J K, Oda K, Tabira T, Shaw I T, Dahrouge S, Antel J P (1992) Neuroblastoma x spinal cord (NSC) hybrid cell lines resemble developing motor neurons. Develop Dynamics 194(3):209-221.

12. Chen F, Hersh B M, Conradt B, Zhou Z, Riemer D, Gruenbaum Y, Horvitz H R (2000) Translocation of C. elegans CED-4 to nuclear membranes during programmed cell death. Science 287(5457):1485-9.

13. Chinnaiyan A M, O'Rourke K, Tewari M, Dixit V M (1995) FADD, a novel death domain-containing protein, interacts with the death domain of Fas and initiates apoptosis. Cell 81(4):505-12.

14. Chow V T K, Lee S S (1996) DENN, a novel human gene differentially expressed in normal and neoplastic cells. DNA Sequence-J Sequencing and Mapping 6:262-273.

15. Chow V T K, Lim K M, Lim D (1998) The human DENN gene: genomic organization, alternative splicing, and localization to chromosome 1 lpl 1.21-pl 1.22. Genome 41:543-552.

16. Citron B A, Zhang S X, Smirnova I V, Festoff B W (1997) Apoptotic, injury-induced cell death in cultured mouse murine motor neurons. Neurosci Lett 230(1):25-8.

17. Cleveland J L, Ihle J N (1995) Contenders in FasL/TNF death signaling. Cell 81:479-482.

18. Coyle J T, Puttfarcken P (1993) Oxidative stress, glutamate, and neurodegenerative disorders. Science 262(5134):689-95.

19. Dickens M, Rogers J S, Cavanagh J, Raitano A, Xia Z, Halpern J R, Greenberg M E, Sawyers C L, Davis R J (1997) A cytoplasmic inhibitor of the JNK signal transduction pathway. Science 277(5326):693-6.

20.Ekinci F J, Linsley M-D, Shea T B (2000) P-Amyloid-induced calcium influx induces apoptosis in culture by oxidative stress rather than tau phosphorylation. Mol Brain Res 76:389-395. Mol Brain Res 76(2):389-95.

21. Eilers A, Whitfield J, Shah B, Spadoni C, Desmond H, Ham J (2001) Direct inhibition of c-Jun N-terminal kinase in sympathetic neurones prevents c-jun promoter activation and NGF withdrawal-induced death. J Neurochem 76(5):1439-54.

22. Estus S, Tucker H M, van Rooyen C, Writhgt S, Brigham E F, Wogulis M, Rydel R E (1997) Aggregated amyloid-beta protein induces cortical neuronal apoptosis and concomitant “apoptotic” pattern of gene induction. J Neurosci 17:7736-7745.

23. Feinstein E, Kimchi A, Wallach D, Boldin M, Varfolomeev E (1995) The death domain: a module shared b proteins with diverse cellular functions. Trends Biochem Sci 20(9):342-4.

24. Gervais F G, Xu D, Robertson G S, Vaillancourt J P, Zhu Y, Huang J, LeBlanc A, Smith D, Rigby M, Shearman M S, Clarke E E, Zheng H, Zamboni R J, Roy S, Nicholson D W (1999) Involvement of caspases in proteolytic cleavage of Alzheimer's amyloid-β precursor protein and amyloidogenic Aβ peptide formation. Cell 97:395-406

25. Gillardon F, Spranger M, Tiesler C, Hossmann K A (1999) Expression of cell death-associated phospho-c-Jun and p53-activated gene 608 in hippocampal CA1 neurons following global ischemia. Brain Res Mol Brain Res 73(1-2):138-43.

26. Giulian D, Haverkamp L J, Yu J H, Karshin W, Tom D, Li J, Kirkpatrick J, Kuo L M, Roher A E (1996) Specific domains of beta-amyloid from Alzheimer plaque elicit neuron killing in human microglia. J Neurosci 16(19):6021-37.

27. Gunn-Moore F J, Tavare J M (1998) Apoptosis of cerebellar granule cells induced by serum withdrawal, glutamate or beta-amyloid, is independent of Jun kinase or p38 mitogen activated protein kinase activation. Neurosci Lett 250(l):53-6.

28. Guo Q, Sebastian L, Sopher B L, Miller M W, Ware C B, Martin, G M, Mattson M P (1999) Increased vulnerability of hippocampal neurons from presenilin-1 mutant knock-in mice to amyloid beta-peptide toxicity: central roles of superoxide production and caspase activation. J Neurochem 72:1019-1029.

29. Han Z, Boyle D L, Aupperle K R, Bennett B, Manning A M, Firestein G S (1999) Jun N-terminal kinase in rheumatoid arthritis. J Pharmacol Exp Ther 291(1): 124-30.

30. Harada J, Sugimoto M (1999) Activation of caspase-3 in β-amyloid-induced apoptosis of cultured rat cortical neurons. Brain Res 842:311-323.

31. Harrington A W, Kim J Y, Yoon S O (2002) Activation of Rac GTPase by p75 is necessary for c-jun N-terminal kinase-mediated apoptosis. J Neurosci 22(1):156-66.

32. Henshall D C, Skradski S L, Bonislawski D P, Lan J Q, Simon R P. Henshall D C, Skradski S L, Bonislawski D P, Lan J Q, Simon R P (2001) Caspase-2 activation is redundant during seizure-induced neuronal death'. J Neurochem 77(3):886-95.

33.Hensley K, Floyd R A, Zheng N Y, Nael R, Robinson K A, Nguyen X, Pye Q N, Stewart C A, Geddes J, Markesbery W R, Patel E, Johnson G V, Bing G (1999) p38 kinase is activated in the Alzheimer's disease brain. J Neurochem 72(5):2053-8.

34. Herdegen T, Claret F X, Kallunki T, Martin-Villalba A, Winter C, Hunter T, Karin M (1998) Lasting N-terminal phosphorylation of c-Jun and activation of c-Jun N-terminal kinases after neuronal injury. J Neurosci 18(14):5124-35.

35. Hsu H, Xiong J, Goeddel D V (1995) The TNF receptor 1-associated protein TRADD signals cell death and NF-kappa B activation. Cell 81(4):495-504.

36. Hsu H, Shu H B, Pan M G, Goeddel D V (1996) TRADD-TRAF2 and TRADD-FADD interactions define two distinct TNF receptor 1 signal transduction pathways. Cell 84(2):299-308.

37. Ii M, Sunamoto M, Ohnishi K, Ichimori Y (1996) beta-Amyloid protein-dependent nitric oxide production from microglial cells and neurotoxicity. Brain Res 720:93-100.

38. Ip Y T, Davis R J (1998) Signal transduction by the c-Jun N-terminal kinase (JNK)-from inflammation to development. Curr Opin Cell Biol 10(2):205-19.

39. Iwasaki K, Staunton J, Saifee O, Nonet M, Thomas J H (1997) aex-3 encodes a novel regulator of presynaptic activity in C. elegans. Neuron 18(4):613-22.

40. Jin K, Mao X O, Zhu Y, Greenberg D A (2002) MEK and ERK protect hypoxic cortical neurons via phosphorylation of Bad. J Neurochem 80(1): 119-25.

41. Johnstone M, Gearing A J, Miller K M (1999) A central role for astrocytes in the inflammatory response to β-amyloid

42. Kruman I, Bruce-Keller A J, Bredesen D, Waeg G, Mattson M P (1997) Evidence that 4-hydroxynonenal mediates oxidative stress-induced neuronal apoptosis. J Neurosci 17(13):5089-100.

43. Kumagae Y, Zhang Y, Kim O J, Miller C A (1999) Human c-Jun N-terminal kinase expression and activation in the nervous system. Mol Brain Res 67:10-17.

44. Le-Niculescu H, Bonfoco E, Kasuya Y, Claret F X, Green D R, Karin M (1999) Withdrawal of survival factors results in activation of the JNK pathway in neuronal cells leading to Fas ligand induction and cell death. Mol Cell Biol 19(1):751-63.

45. Li Y P, Bushnell A F, Lee C M, Perlmutter L S, Wong S K (1996) Beta-amyloid induces apoptosis in human-derived neurotypic SH-SY5Y cells. Brain Res 738(2):196-204.

46. Linnik M D, Hatfield M D, Swope M D, Ahmed N K (1993) Induction of programmed cell death in a dorsal root ganglia X neuroblastoma cell line. J Neurobiol 24(4):433-46.

47. Loo D T, Copani A, Pike C J, Whittemore E R, Walencewicz A J, Cotman C W (1993) Apoptosis is induced by beta-amyloid in cultured central nervous system neurons. PNAS 90(17):7951-5.

48. Maroney A C, Finn J P, Bozyczko-Coyne D, O'Kane T M, Neff N T, Tolkovsky A M, Park D S, Yan C Y, Troy C M, Greene L A (1999) CEP-1347 (KT7515), an inhibitor of JNK activation, rescues sympathetic neurons and neuronally differentiated PC12 cells from death evoked by three distinct insults. J Neurochem 73(5):1901-12.

49. Marquez-Sterling N R, Lo A C, Sisodia S S, Koo E H (1997) Trafficking of cell-surface beta-amyloid precursor protein: evidence that a sorting intermediate participates in synaptic vesicle recycling. J Neurosci 17(1):140-51.

50. Mielke K, Damm A, Yang D D, Herdegen T (2000) Selective expression of JNK isoforms and stress-specific JNK activity in different neural cell lines. Mol Brain Res 75:128-137.

51. Mirra S S, Heyman A, McKeel D, et al. (1991) The consortium to establish a registry for Alzheimer's disease (CERAD). Part II. Standardization of the neuropathologic assessment of Alzheimer's disease. Neurology 41:479-86.

52. Mohit A, Martin J H, Miller C A (1995) p49^{3F12} kinase: A novel MAP kinase expressed in a subset of neurons in the human nervous system. Neuron 14:67-78.

53. Morishima Y, Gotoh Y, Zieg J, Barrett T, Takano H, Flavell R, Davis R J, Shirasaki Y, Greenberg M E (2001) Beta-amyloid induces neuronal apoptosis via a mechanism that involves the c-Jun N-terminal kinase pathway and the induction of Fas ligand. J Neurosci 21(19):7551-60.

54. Murakami-Mori K, Mori S, Bonavida B, Nakamura S (1999) Implication of TNF receptor-I-mediated extracellular signal-regulated kinases 1 and 2 (ERK1/2) activation in growth of AIDS-associated Kaposi's Sarcoma cells: a possible role of a novel death domain protein MADD in TNF-α-induced ERK1/2 activation in Kaposi's Sarcoma cells. J Immunol 162(6):3672-9.

55. Oishi H, Sasaki T, Nagano F, Ikeda W, Ohya T, Wada M, Ide N, Nakanishi H, Takai Y (1998) Localization of the Rab3 small G protein regulators in nerve terminals and their involvement in Ca²⁺ dependent exocytosis. J Biol Chem 273(51):34580-34585.

56. Pedersen W A, Cashman N R, Mattson M P (1999) The lipid peroxidation product 4-hydroxynonenal impairs glutamate and glucose transport and choline acetyltransferase activity in NSC-19 motor neuron cells. Exp Neurol 155(1):1-10.

57. Perini G, Della-Bianca V, Politi V, Della Valle G, Dal-Pra I, Rossi F, Armato U (2002) Role of p75 neurotrophin receptor in the neurotoxicity by beta-amyloid peptides and synergistic effect of inflammatory cytokines. J Exp Med 195(7):907-18.

58. Pettmann B, Henderson C E (1998) Neuronal cell death. Neuron 20(4):633-47.

59. Pike, C J, Walencewicz A J, Glabe C G, Cotman C W (1991) In vitro aging of beta-amyloid protein causes peptide aggregation and neurotoxicity. Brain Res 563:311 -14.

60. Pike C J, Ramezan-Arab N, Cotman C W (1997) Beta-amyloid neurotoxicity in vitro: evidence of oxidative stress but not protection by antioxidants. J Neurochem 69:1601-11.

61. Posmantur R, McGinnis K, Nadimpalli R, Gilbertsen R B, Wang K K W (1997) Characterization of CPP32-like protease activity following apoptotic challenge in SH-SY5Y neuroblastoma cells. J Neurochem 68:2328-2337.

62. Pyo H, Jou I, Jung S, Hong S, Joe E H (1998) Mitogen-activated protein kinases activated by lipopolysaccharide and beta-amyloid in cultured rat microglia. Neuroreport 9(5):871-4.

63. Rimon G, Bazenet C E, Philpott K L, Rubin L L (1997) Increased surface phosphatidylserine is an early marker of neuronal apoptosis. J Neurosci Res 48(6):563-70.

64. Schievella A R, Chen J H, Graham J R, Lin L-L (1997) MADD, a novel death domain protein that interacts with the type 1 tumor necrosis factor receptor and activates mitogen-activated protein kinase. J of Biol Chem 272(18):12069-12075.

65. Schutte B, Nuydens R, Geerts H, Ramaekers F (1998) Annexin V binding assay as a tool to measure apoptosis in differentiated neuronal cells. J Neurosci Methods 86(1):63-9.

66. Selkoe D J (1997) Alzheimer's disease: genotypes, phenotypes and treatments. Science 275(5300):630-1.

67. Selkoe D J (1999) Translating cell biology into therapeutic advances in Alzheimer's disease. Nature 399(6738 Suppl):A23-31.

68. Schneider P, Tschopp J (2000) Apoptosis induced by death receptors. Pharm Acta Helv 74(2-3):281-6.

69. Smirnova I V, Citron B A, Arnold P M, Zhang S X, FestoffBW (1998) Characterization of apoptosis in a motor neuron cell line. Spine 23(2):151-158.

70. Stefanis L, Park D S, Friedman W J, Greene L A (1999) Caspase-dependent and -independent death of camptothecin-threated embryonic cortical neurons. J of Neuroscience 19(15):6235-6247.

71. Stegh A H, Schickling O, Ehret A, Scaffidi C, Peterhansel C, Hofmann A, Grummt I, Krammer P H, Peter M E (1998) DEDD, a novel death effector domain-containing protein, targets to the nucleolus. EMBO J 17(20):5974-86.

72. The National Institute on Aging, and Reagan Institute Working Group on diagnostic criteria for the neuropathological assessment of Alzheimer's disease. (1997) Consensus recommendations for the post-mortem diagnosis of Alzheimer's disease. Neurobiol Aging 18(S4):S1-S2.

73. Toumier C, Hess P, Yang D D, Xu J, Turner T K, Nimnual A, Bar-Sagi D, Jones S N, Flavell R A, Davis R J (2000) Requirement of JNK stress-induced activation of the cytochrome c-mediated death pathway. Science 288:870-74.

74. Troy C M, Rabacchi S A, Friedman W J, Frappier T F, Brown K, Shelanski M L (2000) Caspase-2 mediates neuronal cell death induced by β-amyloid. J of Neuroscience 20(4): 1386-92.

75. Troy C M, Rabacchi S A, Xu Z, Maroney A C, Connors T J, Shelanski M L, Greene L A (2001) beta-Amyloid-induced neuronal apoptosis requires c-Jun N-terminal kinase activation. J Neurochem 77(1):157-64.

76. Uetsuki T, Takemoto K, Nishimura I, Okamoto M, Niinobe M, Momoi T, Miura M, Yoshikawa K (1999) Activation of neuronal caspase-3 by intracellular accumulation of wild-type Alzheimer amyloid precursor protein. J of Neuroscience 19(16):6955-6964.

77. Wada M, Nakanishi H, Satoh A, Hirano H, Obaishi H, Matsuura Y, Takai Y (1997) Isolation and characterization of a GDP/GTP exchange protein specific for the Rab3 subfamily small G proteins. J Biol Chem 272(7):3875-3878.

78. Watt J A, Pike C J, Walencewicz-Wasserman A J, Cotman C W (1994) Ultrastructural analysis of beta-amyloid-induced apoptosis in cultured hippocampal neurons. Brain Res 661 (1 -2): 147-56.

79. Wallach D, Varfolomeev E E, Malinin N L, Goltsev Y V, Kovalenko A V, Boldin M P (1999) Tumor necrosis factor receptor and Fas signaling mechanisms. Annu Rev Immunol 17:331-67.

80. Whitmarsh A J, Kuan C Y, Kennedy N J, Kelkar N, Haydar T F, Mordes J P, Appel M, Rossini A A, Jones S N, Flavell R A, Rakic P, Davis R J (2001) Requirement of the JIP1 scaffold protein for stress-induced JNK activation. Genes Dev 15(18):2421-32.

81. Xia Z, Dickens M, Raingeaud J, Davis R J, Greenberg M E (1995) Opposing effects of ERK and JNK-p38 MAP kinases on apoptosis. Science 270(5240):1326-31.

82. Yan S D, Roher 'A, Chaney M, Zlokovic B, Schmidt A M, Stem D (2000) Cellular cofactors potentiating induction of stress and cytotoxicity by amyloid β-peptide. Biochim Biophys Acta 1502(1):145-157.

83. Yang D D, Kuan C Y, Whitmarsh A J, Rincon M, Zheng T S, Davis R J, Rakic P, Flavell R A (1997) Absence of excitotoxicity-induced apoptosis in the hippocampus of mice lacking the Jnk3 gene. Nature 389(6653):865-70.

84. Yankner B A (1996) New clues to Alzheimer's disease: unraveling the roles of amyloid and tau. Nat Med 2(8):850-2.

85. Yuan J, Yankner B A (2000) Apoptosis in the nervous system. Nature 407(6805):802-9.

86. Zhang Y, Zhou L, Miller C A (1998) A splicing variant of a death domain protein that is regulated by a mitogen-activated kinase is a substrate for c-Jun N-terminal kinase in the human central nervous system. PNAS 95:2586-2591.

87. Zhu X, Castellani R J, Takeda A, Nunomura A, Atwood C S, Perry G, Smith M A (2001) Differential activation of neuronal ERK, JNK/SAPK and p38 in Alzheimer disease: the ‘two hit’ hypothesis. Mech Ageing Dev 123(1):39-46.

What is claimed is:

1. A method of treating a neurological disorder in a subject, the method comprising: administering to said subject an effective amount of a composition for inhibiting the interaction between DENN-MADD and a JNK.

2. The method of claim 1 wherein the composition comprises a compound that inhibits the activity of JNK.

3. The method of claim 2 wherein said JNK is selected from the group consisting of JNK1, JNK2, JNK3 and isoforms thereof.

4. The method of claim 2 wherein said compound is a pyridyl imidazole compound.

5. The method of claim 4 wherein said pyridyl imidazole compound is 4-(4-fluorophenyl)-2-(4-methlysulfinylphenyl)-5-(4-pyridyl)-1 H-imidazole.

6. The method of claim 2 wherein said compound is an indolocarbazole.

7. The method of claim 6 wherein said indolocarbazole is indolocarbazole JNK inhibitor CEP11004.

8. The method of claim 2 wherein said compound is a DENN antisense nucleotide.

9. The method of claim 8 wherein the DENN antisense oligonucleotide is an oligonucleotide having a sequence substantially equivalent to SEQ ID NO:1, SEQ ID NO:2, SEQ ID NO:3, SEQ ID NO:4, or SEQ ID NO:5.

10. The method of claim 1 wherein the composition comprises a pharmaceutically acceptable carrier.

11. The method of claim 1 wherein the composition is administered orally, transdermally, intravenously, intrasynovially, intramuscularly, intraocularly, intranasally, intrathecally, or topically.

12. The method of claim 1 wherein administering the composition is in conjunction with another method of treating said neurological disorder.

13. The method of claim 1, wherein the neurological disorder is caused by oxidative stress response in neuronal tissue.

14. The method of claim 1, wherein the interaction between DENN-MADD and JNK3 increases the activity of JNK3.

15. The method of claim 1 wherein the neurological disorder is a disorder selected from dementia, dementia of the Alzheimer's type, bipolar disorders, mood disorder with depressive features, mood disorder with major depressive-like episode, mood disorder with manic features, mood disorder with mixed features, substance-induced mood disorder and mood disorder not otherwise specified (NOS), panic disorder without agoraphobia, panic disorder with agoraphobia, agorathobia without history of panic disorder, social phobia, postraumatic stress disorder, acute stress disorder, substance-induced anxiety disorder and anxiety disorder not otherwise specified (NOS), dyskinesias and behavioral manifestations of mental retardation, conduct disorder and autistic disorder.

16. The method of claim 15, wherein dementia is selected from the group consisting of vascular dementia, dementia due to HIV disease, dementia due to head trauma, dementia due to Parkinson's disease, dementia due to Huntington's disease, dementia due to Pick's disease, dementia due to Creutzfeldt-Jakob disease, substance-induced persisting dementia, dementia due to multiple etiologies and dementia not otherwise specified (NOS).

17. The method of claim 15, wherein said dementia is dementia of the Alzheimer's type.

18. The method of claim 17, wherein dementia of the Alzheimer's type is selected from the group consisting of dementia of the Alzheimer's type with early onset uncomplicated, dementia of the Alzheimer's type with early onset with delusions, dementia of the Alzheimer's type with early onset with depressed mood, dementia of the Alzheimer's type with late onset uncomplicated, dementia of the Alzheimer's type with late onset with delusions and dementia of the Alzheimer's type with late onset with depressed mood.

19. The method of claim 1, wherein the composition is administered in a targeted drug delivery system.

20. The method of claim 19, wherein the targeted drug delivery system is a liposome coated with an antibody that specifically targets neuronal tissue.

21. A method of treating Alzheimer's disease, stroke, amyotrophic lateral sclerosis, age associated memory impairment or Parkinson's disease in a human subject, the method comprising administering to said human an effective amount of a composition comprising an oligonucleotide having a sequence that is substantially equivalent to SEQ ID NO: 1, SEQ ID NO:2, SEQ ID NO:3, SEQ ID NO:4, or SEQ ID NO:5.

22. The method of claim 21, wherein the composition is administered to the subject's cells using a recmobinant expression vector that comprises a sequence substantially equivalent to SEQ ID NO: 1, SEQ ID NO:2, SEQ ID NO:3, SEQ ID NO:4, or SEQ ID NO:5.

23. The method of claim 22, wherein administering the composition further comprises:
removing stem cells from a subject's bone marrow;
introducing the recombinant expression vector into the removed stem cells; and
re-introducing the stem cells into the subject's bone marrow.

24. A method of treating a neurological disease in a human subject selected from the group consisting of Alzheimer's disease, stroke, amyotrophic lateral sclerosis, age associated memory impairment and Parkinson's disease, the method comprising administering to said human an effective amount of a composition comprising a polypeptide having a sequence that is substantially equivalent to SEQ ID NO: 2.

25. The method of claim 25 wherein the composition further comprises a pharmaceutically acceptable carrier.

26. The method of claim 25 wherein the composition is administered orally, transdermally, intravenously, intrasynovially, intramuscularly, intraocularly, intranasally, intrathecally, or topically.

27. The method of claim 25 wherein the method is used in conjunction with another method of treating said neurological disorder.

28. A method of treating a neurological disorder in a subject, the method comprising: administering to said subject an effective amount of a composition for modulating the expression of DENN-MADD.

29. The method of claim 28 wherein the composition comprises a DENN antisense oligonucleotide.

30. The method of claim 29 wherein the DENN antisense oligonucleotide is an oligonucleotide having a sequence substantially equivalent to SEQ ID NO: 1, SEQ ID NO:2, SEQ ID NO:3, SEQ ID NO:4, or SEQ ID NO:5.
